(19) United States
(12) Patent Application Publication
(10) Pub. No.: US 2006/0089321 A1
(43) Pub. Date: Apr. 27, 2006

(54) Cks1 inhibitors

(76) Inventors: Annette O Walter, Mill Valley, CA (US); Christoph Reinhard, Alameda, CA (US); Anne B. Jefferson, Oakland, CA (US); Blanche-Marie F. Shamoon, Berkely, CA (US)

Correspondence Address: Lisa E Alexander;Chiron Corporation, Intellectual Property, P O Box 8097, Emeryville, CA, 94662-8097, US

(21) Appl. No.: 10/504,487

(22) PCT Filed: Feb. 12, 2003
(86) PCT No.: PCT/US03/04550

§ 371 Date: Oct. 19, 2005

Related U.S. Application Data
(60) Provisional application No. 60/356,903, filed on Feb. 12, 2002.

(51) Int. Cl. A61K 48/00

(52) U.S. Cl. 514/44

(57) ABSTRACT

Inhibitors of human Cks1 and human Skp2, including antisense oligonucleotides, methods, and compositions specific for human Cks1 and human Skp2, are provided. Methods of using the compositions for modulating Cks1 expression and Skp2 expression, and for regulating cell growth, particularly tumor cell growth, are also provided.

### BACKGROUND OF THE INVENTION

1. Technical Field

The present invention provides methods and compositions for modulating the expression of Cks1 and Skp2, and antisense and ribozyme compounds specifically hybridizable with Cks1 or Skp2.

2. Description of the Related Art

Cks proteins are low molecular weight (9-18 kDa) ubiquitously expressed proteins that directly bind to cyclin-dependent kinase (CDK)/cyclin complexes. They were initially identified as mutants that are able to genetically suppress defective alleles of CDKs in yeast. Human Cks1 and Cks2 were cloned 1990 and most of the data indicated a function during mitosis like proteasomal degradation of ubiquitinated cyclin B. Recently, Cks1 was shown to be not essential in mice and Cks1−/− cells proliferate more slowly but do not arrest in mitosis. At the same time Cks1 was shown to associate with p45Skp2, a subunit of the SCF ubiquitin ligase which mediates degradation of several cell cycle regulators in G1/S-phase. Analysis of the Cks−/− cells revealed that depletion of Cks1 induced an increase in cyclin E and p27Kip1 protein levels, indicating that Cks1 indeed plays a role in degradation during G1/S-phase, a function independent of CDKs.

Skp2 has been implicated in the ubiquitin-mediated degradation of regulators of mammalian G1 progression, including the cyclin-dependent kinase p27. p27 is a tumor suppressor protein that works in a dosage-dependent manner. (Fero, M. L. et al., Nature (1998) 396:177-180).

Due to its role in the cell cycle, with implications for a role in cancer, there is a need in the art for compositions and methods that regulate expression and/or function of Cks1 and the associated protein Skp2.

### SUMMARY OF THE INVENTION

The present invention provides, in one embodiment, inhibitors of Cks1 and Skp2. Inventive inhibitors include, but are not limited to, antisense molecules, ribozymes, antibodies or antibody fragments, proteins or polypeptides as well as small molecules. Exemplary antisense molecules comprise at least 10, 15 or 20 consecutive nucleotides of or hybridize under stringent conditions to the nucleic acid of SEQ ID NO:1. More preferred are antisense molecules that comprise at least 25 consecutive nucleotides of or hybridize under stringent conditions to the sequence of SEQ ID NO: 1. Representative antisense molecules are provided herein as SEQ ID NOS:26-30.

In further embodiments, compositions are provided that comprise one or more Cks1 or Skp2 inhibitor in a pharmaceutically acceptable carrier.

Additional embodiments provide methods of decreasing Cks1 or Skp2 gene expression or biological activity.

The invention provides an antisense oligonucleotide comprising at least one modified internucleoside linkage.

The invention further provides an antisense oligonucleotide having a phosphorothioate linkage.

The invention still further provides an antisense oligonucleotide comprising at least one modified sugar moiety.

The invention also provides an antisense oligonucleotide comprising at least one modified sugar moiety which is a 2′-O-methyl sugar moiety.

The invention further provides an antisense oligonucleotide comprising at least one modified nucleobase.

The invention still further provides an antisense oligonucleotide having a modified nucleobase wherein the modified nucleobase is 5-methylcytosine.

The invention also provides an antisense compound wherein the antisense compound is a chimeric oligonucleotide.

The invention provides a method of inhibiting the expression of human Cks1 or Skp2 in human cells or tissues comprising contacting the cells or tissues in vivo with an antisense compound or a ribozyme of 8 to 35 nucleotides in length targeted to a nucleic acid molecule encoding human Cks1 or Skp2 so that expression of human Cks1 or Skp2 is inhibited.

The invention further provides a method of modulating growth of cancer cells comprising contacting the cancer cells in vivo with an antisense compound or ribozyme of 8 to 35 nucleotides in length targeted to a nucleic acid molecule encoding human Cks1 or Skp2 so that expression of human Cks1 or Skp2 is inhibited.

The invention still further provides for identifying target regions of Cks1 or Skp2 polynucleotides. The invention also provides labeled probes for identifying Cks1 or Skp2 polynucleotides by in situ hybridization.

The invention provides for the use of aft Cks1 or Skp2 inhibitor according to the invention to prepare a medicament for modulating cell proliferation.

The invention also provides a pharmaceutical composition for inhibiting expression of the Cks1 or Skp2, comprising an antisense oligonucleotide according to the invention in a mixture with a physiologically acceptable carrier or diluent.

The invention further provides a ribozyme capable of specifically cleaving Cks1 or Skp2 RNA, and a pharmaceutical composition comprising the ribozyme.

The invention also provides small molecule inhibitors of Cks1 wherein the inhibitors are capable of reducing the activity of Cks1 or Skp2 or of reducing or preventing the expression of Cks1 or Skp2 mRNA.

The invention therefore provides an isolated Cks1 inhibitor selected from the group consisting of an antisense oligonucleotide, a ribozyme, a protein, a polypeptide, an antibody, and a small molecule.

In a specific embodiment, the isolated Cks1 inhibitor is an antisense molecule.

In a more specific embodiment, the isolated Cks1 inhibitor antisense molecule or the complement thereof comprises at least 10 consecutive nucleic acids of the sequence of SEQ ID NO:1.

In another specific embodiment, the isolated Cks1 inhibitor antisense molecule or the complement thereof hybridizes under high stringency conditions to the sequence of SEQ ID NO:1.

In specific embodiments, the isolated Cks1 inhibitor antisense molecule comprises a nucleic acid sequence selected from the group consisting of SEQ ID NO:26-30.

In another embodiment, the isolated Cks1 inhibitor is a ribozyme, and in yet other embodiments, the isolated Cks1 inhibitor is selected from the group consisting of an antibody and an antibody fragment.

The invention further provides a composition comprising a therapeutically effective amount of a Cks1 inhibitor in a pharmaceutically acceptable carrier.

In certain embodiments, this composition comprises two or more Cks1 inhibitors in the composition, and the Cks1 inhibitor is an antisense molecule.

In specific embodiments of the composition, the antisense molecule or the complement thereof comprises at least 10 consecutive nucleic acids of the sequence of SEQ ED NO:1, and in more specific embodiments of the composition, the antisense molecule comprises a nucleic acid sequence selected from the group consisting of SEQ ID NO:26-30.

The invention yet further provides a method of inhibiting the expression of Cks1 in a mammalian cell, comprising administering to said cell an Cks1 inhibitor selected from the group consisting of an antisense oligonucleotide, a ribozyme, a protein, a polypeptide, an antibody, and a small molecule.

In certain embodiments of this method, the Cks1 inhibitor is an antisense molecule.

The invention still further provides a method of inhibiting the expression of Cks1 gene expression in a subject, comprising administering to said subject, in a pharmaceutically effective vehicle, an amount of an antisense oligonucleotide which is effective to specifically hybridize to all or part of a selected target nucleic acid sequence derived from said Cks1 gene.

In certain embodiments of this method, the antisense oligonucleotide is selected from the group consisting of SEQ ID NO:26-30.

The invention also provides a method of treating neoplastic disease, comprising administering to a mammalian cell an Cks1 inhibitor selected from the group consisting of an antisense oligonucleotide, a ribozyme, a protein, a polypeptide, an antibody, and a small molecule such that the neoplastic disease is reduced in severity.

Other embodiments provide an antisense compound of 8 to 35 nucleotides in length targeted to a nucleic acid molecule encoding human Cks1, wherein the antisense compound inhibits the expression of human Cks1, and an isolated polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOS:26-30.

Also provided is a recombinant vector comprising a polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOS:26-30.

The invention therefore provides an isolated Skp2 inhibitor selected from the group consisting of an antisense oligonucleotide, a ribozyme, a protein, a polypeptide, an antibody, and a small molecule.

In a specific embodiment, the isolated Skp2 inhibitor is an antisense molecule.

In a more specific embodiment, the isolated Skp2 inhibitor antisense molecule or the complement thereof comprises at least 10 consecutive nucleic acids of the sequence of SEQ ID NO:3.

In another specific embodiment, the isolated Skp2 inhibitor antisense molecule or the complement thereof hybridizes under high stringency conditions to the sequence of SEQ ID NO:3.

In specific embodiments, the isolated Skp2 inhibitor antisense molecule comprises a nucleic acid sequence selected from the group consisting of SEQ ID NO:5, 7, 9, 11, 13, 15, 17, 19, 21 and 23.

In another embodiment, the isolated Skp2 inhibitor is a ribozyme, and in yet other embodiments, the isolated Skp2 inhibitor is selected from the group consisting of an antibody and an antibody fragment.

The invention further provides a composition comprising a therapeutically effective amount of a Skp2 inhibitor in a pharmaceutically acceptable carrier.

In certain embodiments, this composition comprises two or more Skp2 inhibitors in the composition, and the Skp2 inhibitor is an antisense molecule.

In specific embodiments of the composition, the antisense molecule or the complement thereof comprises at least 10 consecutive nucleic acids of the sequence of SEQ ID NO:25, and in more specific embodiments of the composition, the antisense molecule comprises a nucleic acid sequence selected from the group consisting of SEQ ID NO:5, 7, 9, 11, 13, 15, 17, 19, 21 and 23.

The invention yet further provides a method of inhibiting the expression of Skp2 in a mammalian cell, comprising administering to said cell a Skp2 inhibitor selected from the group consisting of an antisense oligonucleotide, a ribozyme, a protein, a polypeptide, an antibody, and a small molecule.

In certain embodiments of this method, the Skp2 inhibitor is an antisense molecule.

The invention still further provides a method of inhibiting the expression of Skp2 gene expression in a subject, comprising administering to said subject, in a pharmaceutically effective vehicle, an amount of an antisense oligonucleotide which is effective to specifically hybridize to all or part of a selected target nucleic acid sequence derived from said Skp2 gene.

In certain embodiments of this method, the antisense oligonucleotide is selected from the group consisting of SEQ ID NO:5, 7, 9, 11, 13, 15, 17, 19, 21 and 23.

The invention also provides a method of treating neoplastic disease, comprising administering to a mammalian cell an Skp2 inhibitor selected from the group consisting of an antisense oligonucleotide, a ribozyme, a protein, a polypeptide, an antibody, and a small molecule such that the neoplastic disease is reduced in severity.

Other embodiments provide an antisense compound of 8 to 35 nucleotides in length targeted to a nucleic acid molecule encoding human Skp2, wherein the antisense compound inhibits the expression of human Skp2, and an isolated polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOS:5, 7, 9, 11, 13, 15, 17, 19, 21 and 23.

Also provided is a recombinant vector comprising a polynucleotide with a sequence comprising a transcriptional initiation region and a sequence encoding an antisense oligonucleotide at least 8 nucleotides or nucleotide analogues and not longer than 35 nucleotides in length comprising a sequence selected from the group consisting of SEQ ID NOS:5, 7, 9, 11, 13, 15, 17, 19, 21 and 23.

### BRIEF DESCRIPTION OF THE DRAWINGS

FIG. 1 is a Cks1 polynucleotide (SEQ ID NO:1).

FIG. 2 is a Skp2 polynucleotide (SEQ ID NO:3) and polypeptide (SEQ ID NO:4).

FIG. 3 is a bar graph showing expression of Cks1 messenger RNA, normalized to actin, in tumor cell lines.

FIG. 4 is a graph showing that Cks1, as well as Cks2, are required for cell proliferation in tumor cells, as shown by treating SW620 cells with antisense oligonucleotides specific for Cks1 (FIG. 4A, SEQ ID NO:30) or Cks2 (FIG. 4B, SEQ ID NO: 39). FIG. 4C shown proliferation of cells treated with SEQ ID NO:15, specific for Skp2.

FIG. 5 is a bar graph showing that depletion of Cks1 inhibits anchorage-independent growth of SW620 cells, using SEQ ID NO:27 and 30.

FIG. 6 is a bar graph showing that Cks1 depletion by antisense treatment caused cytotoxicity in MRC9 (FIG. 6A) cells and SW620 (FIG. 6B) cells.

FIG. 7A is a bar graph showing that inhibition of Cks1 expression using an antisense oligonucleotide results in accumulation of p27Kip1. FIG. 7B is a photograph confirming the accumulation of p27Kip1. p34cdc2 is a cell cycle dependent kinase, also known as Ckd1. p45Skp2 is protein Skp2.

FIG. 8 shows that depletion of Cks1 but not Cks2 affects p27Kip1 protein levels in tumor cells.

FIG. 9 shows that Cks1 depletion by antisense treatment does not significantly alter the cell cycle profile of asynchronously growing SW620 and MRC9 cells.

FIG. 10 shows that Skp2 depletion did not affect cyclin E degradation directly. Skp2AS is SEQ ID NO:15 and Skp2RC is SEQ ID NO:16. In FIG. 10A, Skp2AS but not Skp2RC decreased Skp2 mRNA levels in treated SW620 colon cancer cells. In FIG. 10B, cyclin E levels are not significantly affected by Skp2AS.

FIG. 11 is a bar graph showing that Skp2AS (SEQ ID NO:15), but not Skp2 RC (SEQ ID NO:16) decreased Skp2 mRNA levels in normal human fibroblasts.

FIG. 12 is a bar graph showing the effect of Cks1 and Skp2 depletion on anchorage independent growth.

FIG. 13 is a bar graph showing that p27 depletion reverses the effect of Cks1 antisense on anchorage-independent growth.

### DETAILED DESCRIPTION OF THE INVENTION

Introduction

The invention relates to the use of inhibitors, preferably oligonucleotides, such as antisense molecules or ribozymes, to target and modulate the expression of polynucleotides comprising a Cks1 nucleotide sequence or a Skp2 nucleotide sequence.

The invention disclosed herein demonstrates that Cks1 plays a pivotal role in tumorgenisis. Depletion of Cks1 in tumor cells using antisense oligonucleotides targeting Cks1 mRNA induced cell death after 2 days, reduced cell proliferation, and inhibited colony formation in soft agar, indicating that Cks1 is required for cell survival, cell proliferation, and anchorage independent growth of tumor cells. Cks1 inhibition did not affect the cell cycle profile of SW620 cells although it induced the accumulation of the CDK inhibitor p27Kip1 as demonstrated by Western blot analysis.

Cks1 directs the ubiquitin-mediated proteolysis of the CDK-bound substrate p27Kip1 by the protein ubiquitin ligase (E3) SCF (Skp2). (Spruck, C. et al., Mol. Cell. 2001 7:639-650.) Cks1 associates with the F box protein Skp2 and is essential for recognition of the p27Kip1 substrate for ubiquitination in vivo and in vitro. p27Kip1 ubiquitination activity is dependent on Cks1. CKS1−/− mice are abnormally small, and cells derived from them proliferate poorly, particularly under limiting mitogen conditions, possibly due to elevated levels of p27Kip1. The invention herein demonstrates that antisense oligonucleotides specific for Skp2 polynucleotides inhibit tumor cell growth as well as induce tumor cell death.

Analysis of a tumor tissue database revealed that Cks1 (CHIR39) mRNA levels are increased in tumors of colon and breast cancer patients compared to normal tissue by 20-40%. Further expression data confirmed overexpression of Cks1 in breast and colon cancer cell lines and tissues.

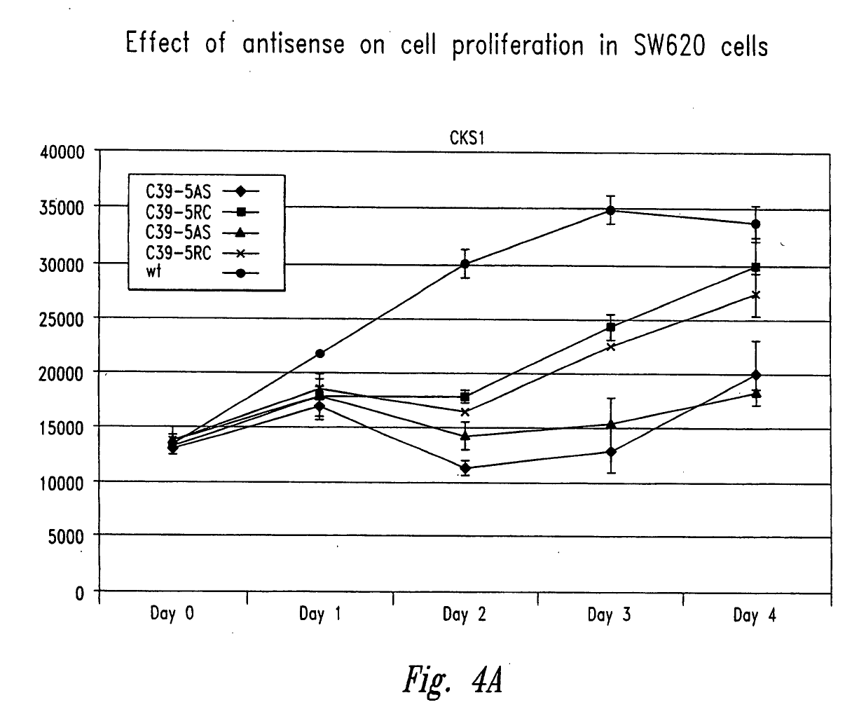

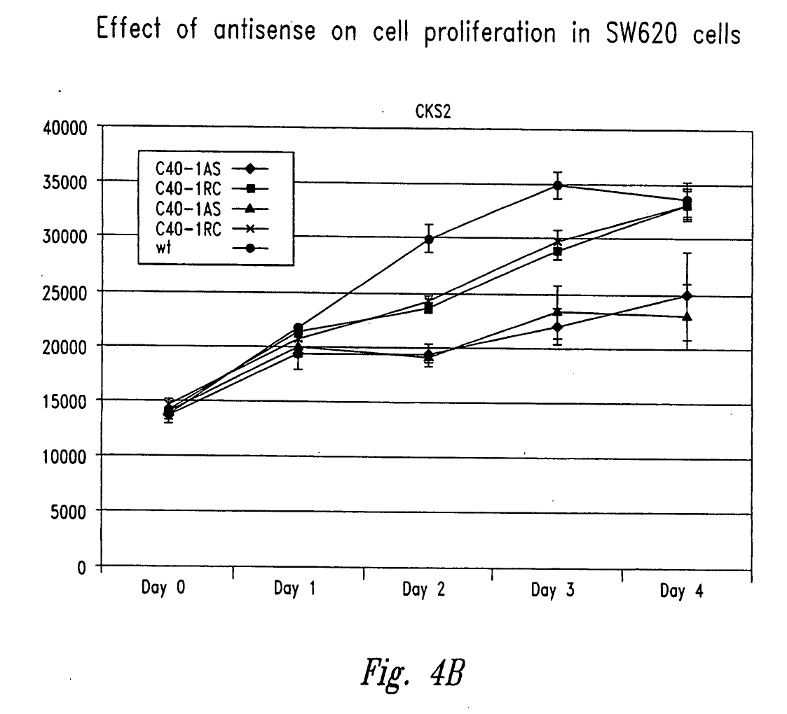

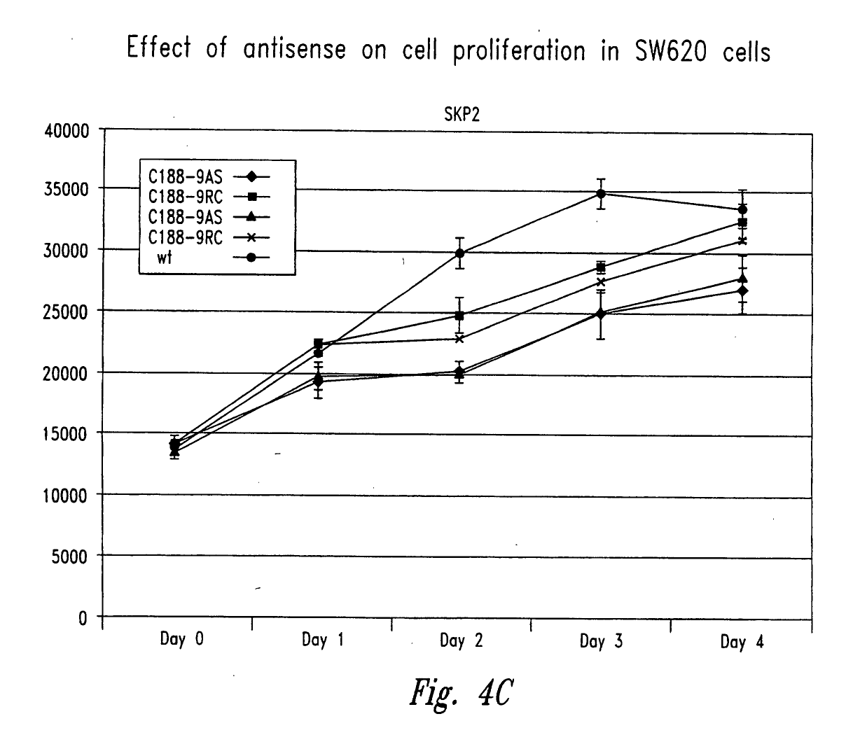

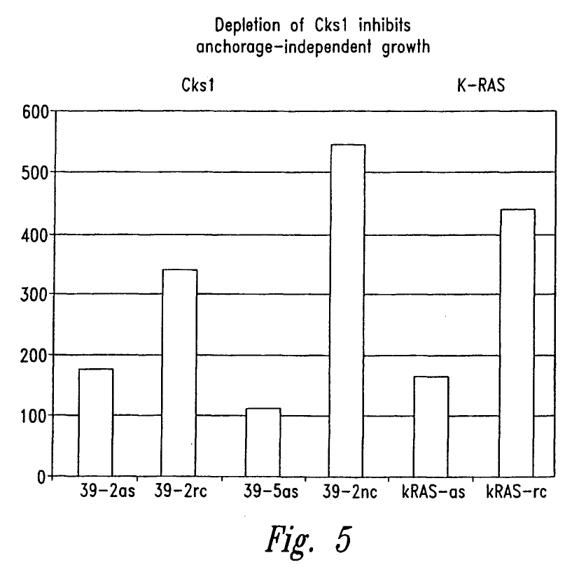

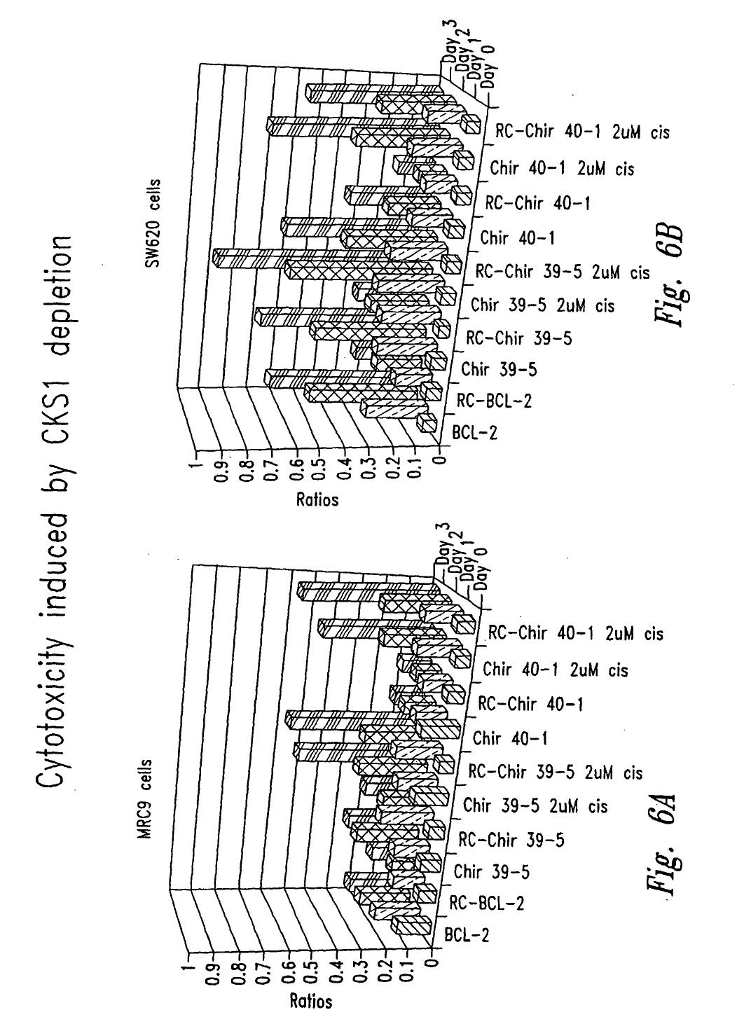

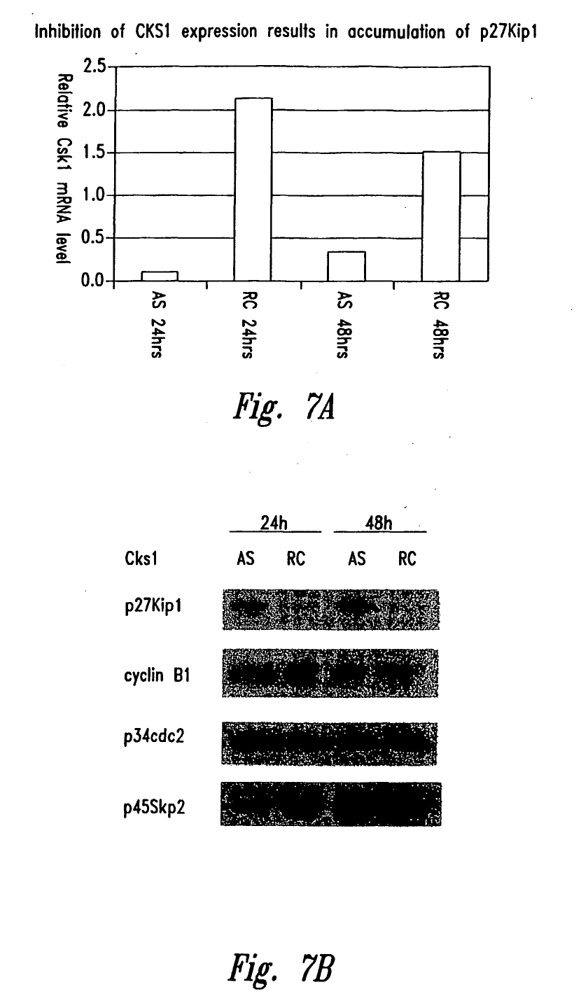

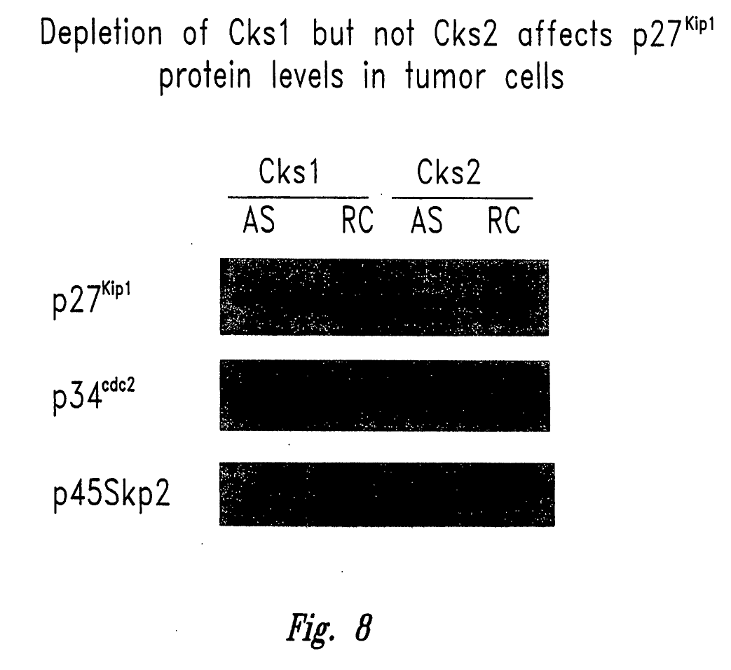

The data disclosed herein also showed that Cks1, and to a lesser extent Cks2, are required for cell proliferation in tumor cells. Treatment of SW620 cells with antisense oligonucleotides specific for Cks1 or Cks2 resulted in inhibition of growth in the Cks1 antisense-treated cells, but not in the Cks2 antisense-treated cells (FIG. 4). Treatment of SW620 cells with Cks1 antisense oligonucleotide also caused an accumulation of p27Kip1 (FIG. 6), but treatment with Cks2 antisense oligonucleotide did not produce this effect. These results confirm previous studies of p27Kip1 levels in Cks1−/− cells from mice.

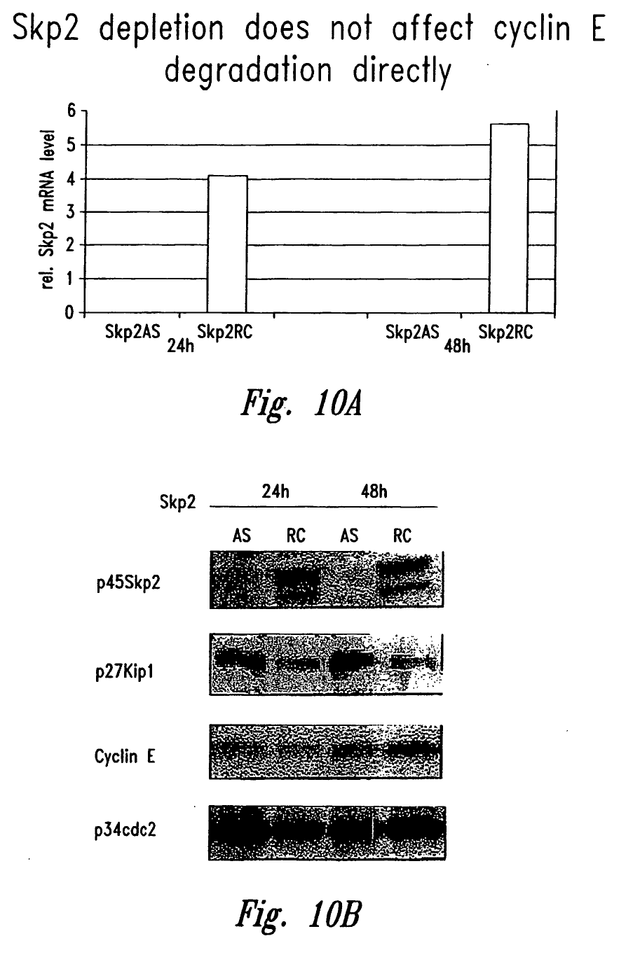

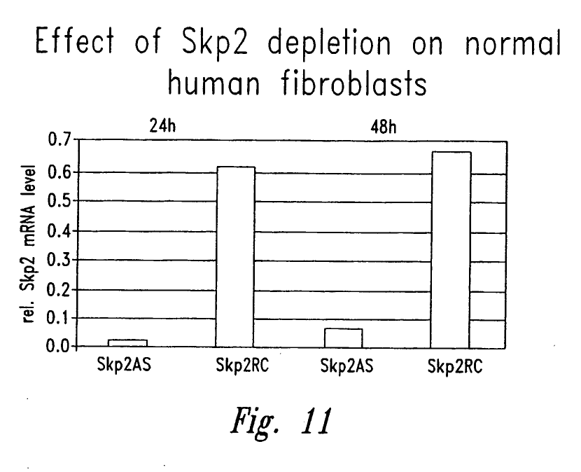

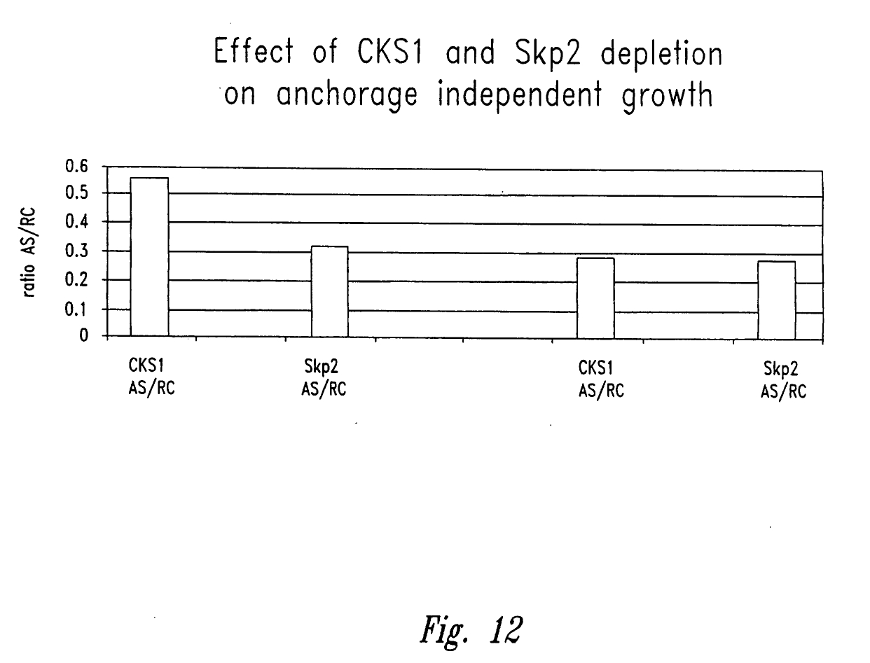

Treatment of normal human fibroblasts and SW620 cells with an antisense oligonucleotide specific for Skp2 (SEQ ID NO: 15) reduced Skp2 mRNA levels, whereas treatment with the corresponding reverse control oligonucleotide (SEQ ID NO:16) did not affect Skp2 mRNA levels (FIGS. 11 and 10A, respectively). Skp2 deletion by treatment with SEQ ID NO:15 inhibited anchorage-independent growth of SW620 cells to some extent, as shown in FIG. 12.

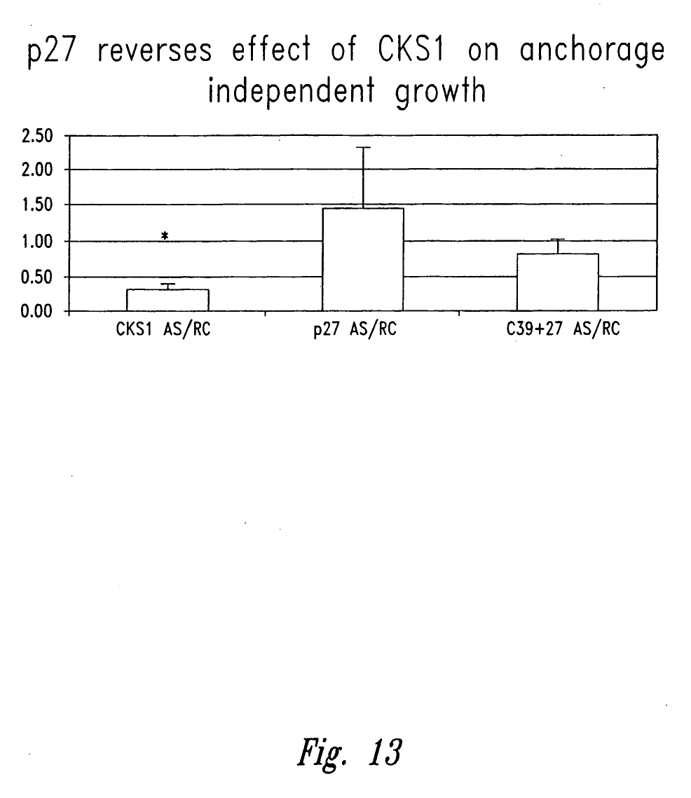

Because of the role played by Skp2 in mediating the tumor suppressor p27, the effect of Skp2 antisense treatment on cyclin E degradation and on p27Kip1 levels was measured. As shown in FIG. 10B, at both 28 and 48 hours, antisense-treated SW620 cells showed higher levels of p27Kip1 than did reverse control-treated cells. Cyclin E protein levels were not significantly altered by Skp2 antisense treatment. p34cdc2, a protein not affected by degradation, served as a gel-loading control. (FIG. 10B). p27 depletion was able to reverse the effect of Cks1 depletion on anchorage-independent growth in SW620 cells, as shown in FIG. 13.

The results herein are consistent with a recent report that Skp2 is oncogenic and expressed in human cancers. Gstaiger, M. et al. (P.N.A.S. 98:5043-5048, 2001) found that increased levels of Skp2 protein were associated with reduced levels of the tumor suppressor p27 in oral epithelial dysplasias and carcinomas compared to normal epithelial controls. The present results add to this information by demonstrating for the first time that inhibition of Skp2 using an antisense oligonucleotide specific for Skp2 can inhibit tumor cells proliferation in vitro. This inhibition appears to be due to the known role of Skp2 in relation to p27 levels, because inhibition of Skp2 is associated with increased levels of p27Kip1 (see FIG. 10B). It is of interest that reduction of p27 protein levels by antisense oligonucleotides has now been shown to reverse the inhibitory effect of Cks1-specific antisense oligonucleotide on anchorage-independent growth of SW620 cells (see FIG. 13), indicating that the inhibition of proliferation by Cks1 is indeed mediated through p27Kip1.

Oligonucleotides for Targeting Cks1 and Skp2 Polynucleotides

According to the present invention, oligonucleotide molecules capable of hybridizing with Cks1 or Skp2 polynucleotides inhibited the proliferation of colon cancer cell line SW620. This cell line is a standard model for cancer cell proliferation and growth in vivo, and the results support in vivo use of the Cks1 and Skp2 antisense molecules to ameliorate cancer in humans and other mammals.

SW620 is an established model system for colon cancer, as recognized by those of skill in the art. These cells are derived from metastasized secondary tumors resected from a single colon cancer patient (Hewitt et al., J. Pathol. (2000) 192:446-54). They are fibroblast-like in appearance and highly tumorigenic; SW620 xenografts form solid sheets of tumor cells. SW620 cells have been used to study the advanced stages of progression of colon cancer (Hewitt et al., J. Pathol. (2000) 192:455-59; Smith et al., J. Nucl. Med (2000) 41:1753-59; O'Connell et al., J. Cell Physiol. (2000) 185:331-38).

The present invention relates to antisense oligonucleotides directed to Cks1 or Skp2 polynucleotides. Antisense strategies directed against the other genes in SW620 cells have been disclosed previously. Transfecting SW620 cells with a plasmid encoding antisense FasL cDNA suggests that impairing FasL translation can inhibit tumor progression (Nyhus et al., Gene Ther. (2001) 8:209-14). p53 antisense oligonucleotides inhibited the growth of SW620 (Hirota et al., Jpn. J. Cancer Res. (1996) 87:635-42), and reducing matrilysin levels in SW620 cells through antisense treatment has been suggested as a method for reducing tumorigenicity and progression of colorectal tumors (Witty et al., Cancer Res. (1994) 54:4805-12). The present invention adds to this knowledge about treating colon cancer cells by disclosing for the first time that antisense oligonucleotides directed against Cks1 are suitable agents for treating colon cancer.

Included within the scope of the invention are oligonucleotides capable of hybridizing with Cks1 DNA or RNA, referred to as the target polynucleotide. An oligonucleotide need not be 100% complementary to the target polynucleotide, as long as specific hybridization is achieved. The degree of hybridization to be achieved is that which interferes with the normal function of the target polynucleotide, be it transcription, translation, pairing with a complementary sequence, or binding with another biological component such as a protein. An antisense oligonucleotide can interfere with DNA replication and transcription, and it can interfere with RNA translocation, translation, splicing, and catalytic activity.

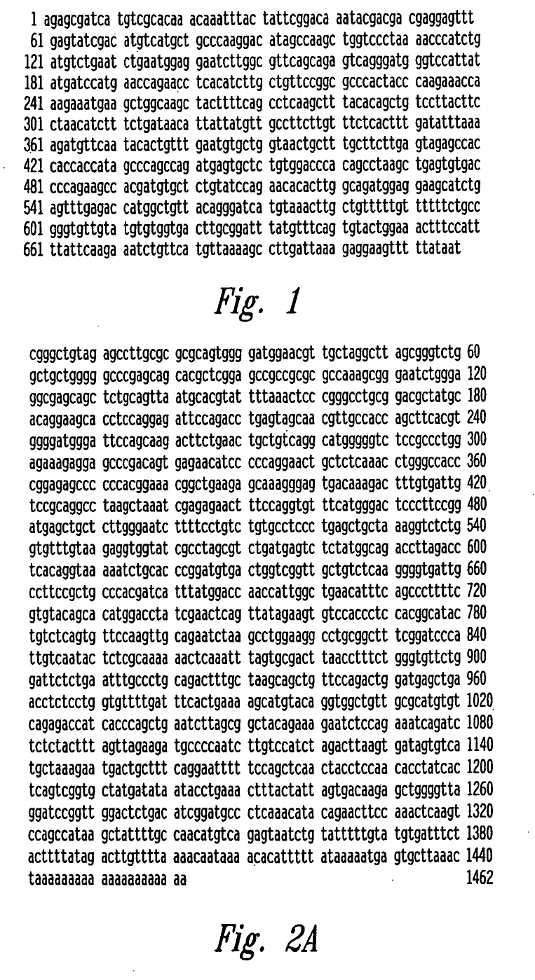

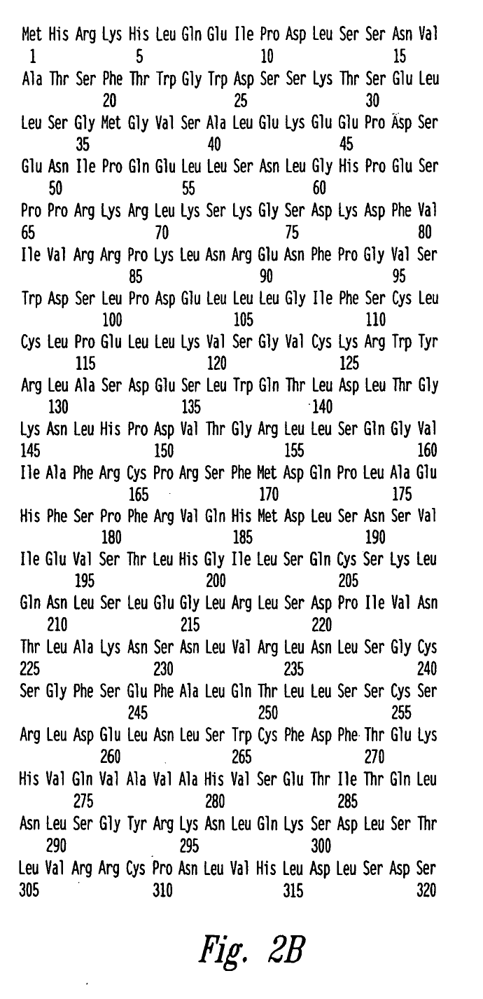

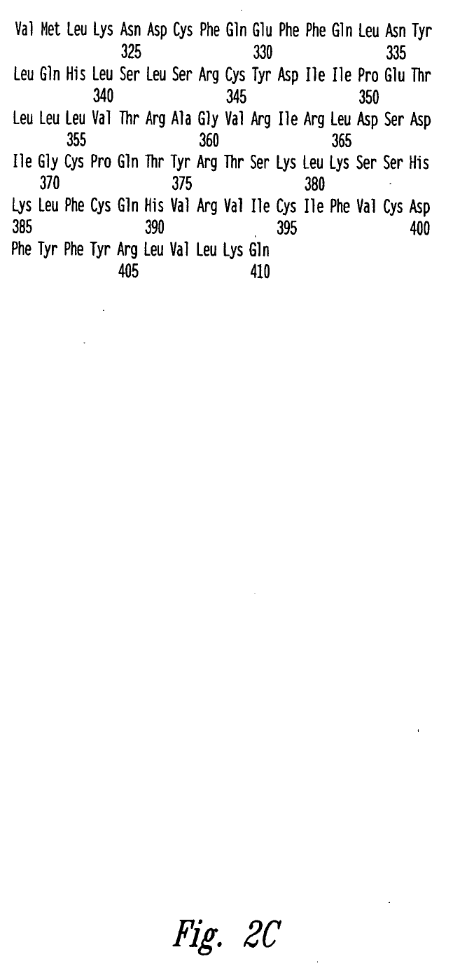

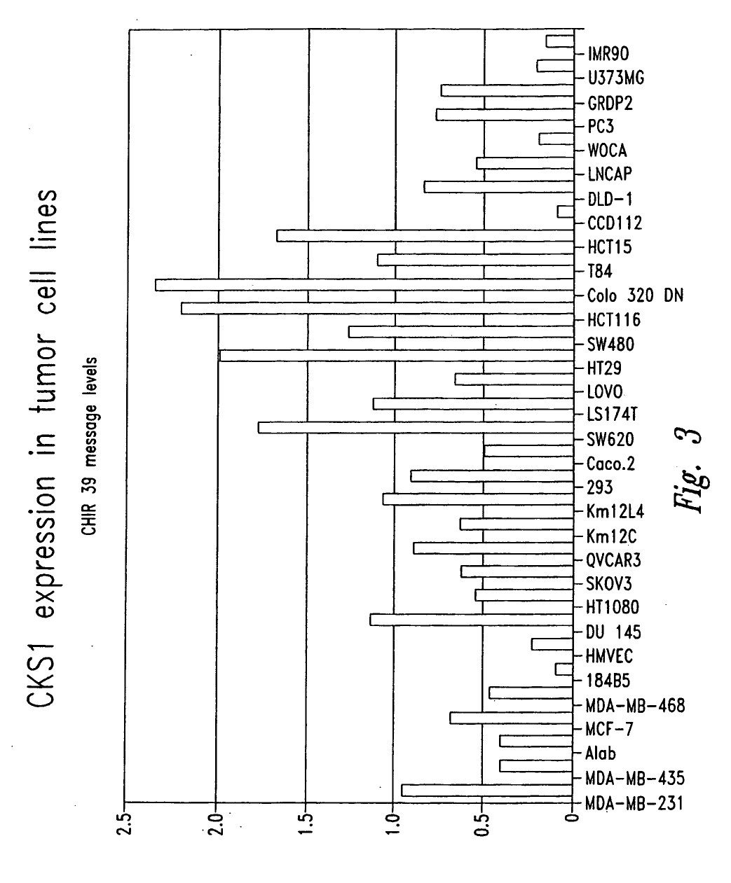

The invention includes within its scope any oligonucleotide of about 8 to about 35 nucleotides in length, including variations as described herein, wherein the oligonucleotide hybridizes to a Cks1 polynucleotide, including DNA or mRNA, such that an effect on the normal function of the polynucleotide is achieved. The oligonucleotide can be 8, 10, 15, 17, 18, 20, 22, 25, 28, 30, 32, or 35 nucleotides in length. The nucleotide sequence of Cks1 is shown in FIG. 1 (SEQ ID NO:1). Preferred antisense oligonucleotides include:

| | | |
|---|---|---|
| CHIR39-1 | CTGCTGAACGCCAAGATTCCTCCAT | SEQ ID NO:26 |
| CHIR39-2 | ACCAGCTTGGCTATGTCCTTGGGC | SEQ ID NO:27 |
| CHIR39-3 | CCCATCCCTGACTCTGCTGAACGC | SEQ ID NO:28 |
| CHIR39-4 | TTTGGTTTCTTGGGTAGTGGGCGC | SEQ ID NO:29 |
| CHIR39-5 | ACTCATCTGGCTGGGCTATGGTGGT | SEQ ID NO:30 |

Control oligonucleotides are as follows:

| | | |
|---|---|---|
| CHIR39-1RC | TACCTCCTTAGAACCGCAAGTCGTC | SEQ ID NO:31 |
| CHIR39-2RC | CGGGTTCCTGTATCGGTTCGACCA | SEQ ID NO:32 |
| CHIR39-3RC | CGCAAGTCGTCTCAGTCCCTACCC | SEQ ID NO:33 |
| CHIR39-4RC | CGCGGGTGATGGGTTCTTTGGTTT | SEQ ID NO:34 |
| CHIR39-5RC | TGGTGGTATCGGGTCGGTCTACTCA | SEQ ID NO:35 |

The antitumor use of the oligonucleotides disclosed herein is based on the discovery that Cks1 antisense oligonucleotides can reduce Cks1 mRNA levels in tumor cells, and can inhibit proliferation of cells of two separate tumor cell lines. To measure the effect on mRNA, MRC9 cells and SW620 cells were incubated with a transfection mixture of an oligonucleotide and a carrier, specifically a lipitoid or cholesteroid, although other carriers can be used as is known in the art. After an incubation of 2-24 hours, the transfection mixture was removed and replaced with normal growth media as described in the Examples.

Total RNA was extracted from the cells, reverse transcribed, and amplified as described in the Examples. Incubation with antisense oligonucleotides (SEQ ID NOS:26-30) reduced the Cks1 mRNA levels relative to actin in MRC9 cells and SW620 cells.

Soft agar assays were performed to determine the effect of the antisense oligonucleotides on anchorage-dependent growth. In both cell lines, colony formation was inhibited by each antisense oligonucleotide. This result demonstrates that inhibition of Cks1 expression also inhibits the ability of tumor cells to grow in an anchorage-independent manner.

Examples of preferred antisense compounds useful in the invention are based on SEQ ID NOS:26-30, and include oligonucleotides containing modified backbones or non-natural internucleoside linkages. Oligonucleotides having modified backbones include those retaining a phosphorus atom in the backbone, and those that do not have a phosphorus atom in the backbone. Preferred modified oligonucleotide backbones include phosphorothioates, chiral phosphorothioates, phosphotriesters, aminoalkylphosphotqiesters, methyl and other alkyl phosphonates including 3′-alkylene phosphonates and chiral phosphonaies, phosphinates, phosphoroamidates including 3′-amino phosphoroamidate and aminoalkylphosphoroamidates, thiophosphoroamidates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′5′ to 5′-2′. Various salts, mixed salts and free acid forms are also included.

Examples of 20-mer oligonucleotides capable of binding to Cks1 polynucleotides include the following oligonucleotides, indicated by polynucleotide positions with reference to SEQ ID NO:1:
1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 726-304, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-1640 146-165, 147-166,.148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201; 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485, 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717.

Examples of 25-mer oligonucleotides capable of binding to Cks1 polynucleotides include the following oligonucleotides, indicated by polynucleotide positions with reference to SEQ ID NO:1:
1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 1943, 20-44, 2145, 22-46, 23A47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 726-309, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 112-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-496, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717.

Examples of 20-mer oligonucleotides capable of binding to Skp2 polynucleotides include the following oligonucleotides, indicated by polynucleotide positions with reference to SEQ ID NO:25:
1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 726-304, 76-95, 77-96, 78-97, 79-98, 80-99, 81-100, 82-101, 83-102, 84-103, 85-104, 86-105, 87-106, 88-107, 89-108, 90-109, 91-110, 92-111, 93-112, 94-113, 95-114, 96-115, 97-116, 98-117, 99-118, 100-119, 101-120, 102-121, 103-122, 104-123, 105-124, 106-125, 107-126, 108-127, 109-128, 110-129, 111-130, 112-131, 113-132, 114-133, 115-134, 116-135, 117-136, 118-137, 119-138, 120-139, 121-140, 122-141, 123-142, 124-143, 125-144, 126-145, 127-146, 128-147, 129-148, 130-149, 131-150, 132-151, 133-152, 134-153, 135-154, 136-155, 137-156, 138-157, 139-158, 140-159, 141-160, 142-161, 143-162, 144-163, 145-164, 146-165, 147-166, 148-167, 149-168, 150-169, 151-170, 152-171, 153-172, 154-173, 155-174, 156-175, 157-176, 158-177, 159-178, 160-179, 161-180, 162-181, 163-182, 164-183, 165-184, 166-185, 167-186, 168-187, 169-188, 170-189, 171-190, 172-191, 173-192, 174-193, 175-194, 176-195, 177-196, 178-197, 179-198, 180-199, 181-200, 182-201, 183-202, 184-203, 185-204, 186-205, 187-206, 188-207, 189-208, 190-209, 191-210, 192-211, 193-212, 194-213, 195-214, 196-215, 197-216, 198-217, 199-218, 200-219, 201-220, 202-221, 203-222, 204-223, 205-224, 206-225, 207-226, 208-227, 209-228, 210-229, 211-230, 212-231, 213-232, 214-233, 215-234, 216-235, 217-236, 218-237, 219-238, 220-239, 221-240, 222-241, 223-242, 224-243, 225-244, 226-245, 227-246, 228-247, 229-248, 230-249, 231-250, 232-251, 233-252, 234-253, 235-254, 236-255, 237-256, 238-257, 239-258, 240-259, 241-260, 242-261, 243-262, 244-263, 245-264, 246-265, 247-266, 248-267, 249-268, 250-269, 251-270, 252-271, 253-272, 254-273, 255-274, 256-275, 257-276, 258-277, 259-278, 260-279, 261-280, 262-281, 263-282, 264-283, 265-284, 266-285, 267-286, 268-287, 269-288, 270-289, 271-290, 272-291, 273-292, 274-293, 275-294, 276-295, 277-296, 278-297, 279-298, 280-299, 281-300, 282-301, 283-302, 284-303, 285-304, 286-305, 287-306, 288-307, 289-308, 290-309, 291-310, 292-311, 293-312, 294-313, 295-314, 296-315, 297-316, 298-317, 299-318, 300-319, 301-320, 302-321, 303-322, 304-323, 305-324, 306-325, 307-326, 308-327, 309-328, 310-329, 311-330, 312-331, 313-332, 314-333, 315-334, 316-335, 317-336, 318-337, 319-338, 320-339, 321-340, 322-341, 323-342, 324-343, 325-344, 326-345, 327-346, 328-347, 329-348, 330-349, 331-350, 332-351, 333-352, 334-353, 335-354, 336-355, 337-356, 338-357, 339-358, 340-359, 341-360, 342-361, 343-362, 344-363, 345-364, 346-365, 347-366, 348-367, 349-368, 350-369, 351-370, 352-371, 353-372, 354-373, 355-374, 356-375, 357-376, 358-377, 359-378, 360-379, 361-380, 362-381, 363-382, 364-383, 365-384, 366-385, 367-386, 368-387, 369-388, 370-389, 371-390, 372-391, 373-392, 374-393, 375-394, 376-395, 377-396, 378-397, 379-398, 380-399, 381-400, 382-401, 383-402, 384-403, 385-404, 386-405, 387-406, 388-407, 389-408, 390-409, 391-410, 392-411, 393-412, 394-413, 395-414, 396-415, 397-416, 398-417, 399-418, 400-419, 401-420, 402-421, 403-422, 404-423, 405-424, 406-425, 407-426, 408-427, 409-428, 410-429, 411-430, 412-431, 413-432, 414-433, 415-434, 416-435, 417-436, 418-437, 419-438, 420-439, 421-440, 422-441, 423-442, 424-443, 425-444, 426-445, 427-446, 428-447, 429-448, 430-449, 431-450, 432-451, 433-452, 434-453, 435-454, 436-455, 437-456, 438-457, 439-458, 440-459, 441-460, 442-461, 443-462, 444-463, 445-464, 446-465, 447-466, 448-467, 449-468, 450-469, 451-470, 452-471, 453-472, 454-473, 455-474, 456-475, 457-476, 458-477, 459-478, 460-479, 461-480, 462-481, 463-482, 464-483, 465-484, 466-485; 467-486, 468-487, 469-488, 470-489, 471-490, 472-491, 473-492, 474-493, 475-494, 476-495, 477-496, 478-497, 479-498, 480-499, 481-500, 482-501, 483-502, 484-503, 485-504, 486-505, 487-506, 488-507, 489-508, 490-509, 491-510, 492-511, 493-512, 494-513, 495-514, 496-515, 497-516, 498-517, 499-518, 500-519, 501-520, 502-521, 503-522, 504-523, 505-524, 506-525, 507-526, 508-527, 509-528, 510-529, 511-530, 512-531, 513-532, 514-533, 515-534, 516-535, 517-536, 518-537, 519-538, 520-539, 521-540, 522-541, 523-542, 524-543, 525-544, 526-545, 527-546, 528-547, 529-548, 530-549, 531-550, 532-551, 533-552, 534-553, 535-554, 536-555, 537-556, 538-557, 539-558, 540-559, 541-560, 542-561, 543-562, 544-563, 545-564, 546-565, 547-566, 548-567, 549-568, 550-569, 551-570, 552-571, 553-572, 554-573, 555-574, 556-575, 557-576, 558-577, 559-578, 560-579, 561-580, 562-581, 563-582, 564-583, 565-584, 566-585, 567-586, 568-587, 569-588, 570-589, 571-590, 572-591, 573-592, 574-593, 575-594, 576-595, 577-596, 578-597, 579-598, 580-599, 581-600, 582-601, 583-602, 584-603, 585-604, 586-605, 587-606, 588-607, 589-608, 590-609, 591-610, 592-611, 593-612, 594-613, 595-614, 596-615, 597-616, 598-617, 599-618, 600-619, 601-620, 602-621, 603-622, 604-623, 605-624, 606-625, 607-626, 608-627, 609-628, 610-629, 611-630, 612-631, 613-632, 614-633, 615-634, 616-635, 617-636, 618-637, 619-638, 620-639, 621-640, 622-641, 623-642, 624-643, 625-644, 626-645, 627-646, 628-647, 629-648, 630-649, 631-650, 632-651, 633-652, 634-653, 635-654, 636-655, 637-656, 638-657, 639-658, 640-659, 641-660, 642-661, 643-662, 644-663, 645-664, 646-665, 647-666, 648-667, 649-668, 650-669, 651-670, 652-671, 653-672, 654-673, 655-674, 656-675, 657-676, 658-677, 659-678, 660-679, 661-680, 662-681, 663-682, 664-683, 665-684, 666-685, 667-686, 668-687, 669-688, 670-689, 671-690, 672-691, 673-692, 674-693, 675-694, 676-695, 677-696, 678-697, 679-698, 680-699, 681-700, 682-701, 683-702, 684-703, 685-704, 686-705, 687-706, 688-707, 689-708, 690-709, 691-710, 692-711, 693-712, 694-713, 695-714, 696-715, 697-716, 698-717, 699-718, 700-719, 701-720, 702-721, 703-722, 704-723, 705-724, 706-725, 707-726, 708-727, 709-728, 710-729, 711-730, 712-731, 713-732, 714-733, 715-734, 716-735, 717-736, 718-737, 719-738, 720-739, 721-740, 722-741, 723-742, 724-743, 725-744, 726-745, 727-746, 728-747, 729-748, 730-749, 731-750, 732-751, 733-752, 734-753, 735-754, 736-755, 737-756, 738-757, 739-758, 740-759, 741-760, 742-761, 743-762, 744-763, 745-764, 746-765, 747-766, 748-767, 749-768, 750-769, 751-770, 752-771, 753-772, 754-773, 755-774, 756-775, 757-776, 758-777, 759-778, 760-779, 761-780, 762-781, 763-782, 764-783, 765-784, 766-785, 767-786, 768-787, 769-788, 770-789, 771-790, 772-791, 773-792, 774-793, 775-794, 776-795, 777-796, 778-797, 779-798, 780-799, 781-800, 782-801, 783-802, 784-803, 785-804, 786-805, 787-806, 788-807, 789-808, 790-809, 791-810, 792-811, 793-812, 794-813, 795-814, 796-815, 701-816, 798-817, 799-818, 800-819, 801-820, 802-821, 803-822, 804-823, 805-824, 806-825, 807-826, 808-827, 809-828, 810-829, 811-830, 812-831, 813-832, 814-833, 815-834, 816-835, 817-836, 818-837, 819-838, 820-839, 821-840, 822-841, 823-842, 824-843, 825-844, 826-845, 827-846, 828-847, 829-848, 830-849, 831-850, 832-851, 833-852, 834-853, 835-854, 836-855, 837-856, 838-857, 839-858, 840-859, 841-860, 842-861, 843-862, 844-863, 845-864, 846-865, 847-866, 848-867, 849-868, 850-869, 851-870, 852-871, 853-872, 854-873, 855-874, 856-875, 857-876, 858-877, 859-878, 860-879, 861-880, 862-881, 863-882, 864-883, 865-884, 866-885, 867-886, 868-887, 869-888, 870-889, 871-890, 872-891, 873-892, 874-893, 875-894, 876-895, 877-896, 878-897, 879-898, 880-899, 881-900, 882-901, 883-902, 884-903, 885-904, 886-905, 887-906, 888-907, 889-908, 890-909, 891-910, 892-911, 893-912, 894-913, 895-914, 896-915, 897-916, 898-917, 899-918, 900-919, 901-920, 902-921, 903-922, 904-923, 905-924, 906-925, 907-926, 908-927, 909-928, 910-929, 911-930, 912-931, 913-932, 914-933, 915-934, 916-935, 917-936, 918-937, 919-938, 920-939, 921-940, 922-941, 923-942, 924-943, 925-944, 926-945, 927-946, 928-947, 929-948, 930-949, 931-950, 932-951, 933-952, 934-953, 935-954, 936-955, 937-956, 938-957, 939-958, 940-959, 941-960, 942-961, 943-962, 944-963, 945-964, 946-965, 947-966, 948-967, 949-968, 950-969, 951-970, 952-971, 953-972, 954-973, 955-974, 956-975, 957-976, 958-977, 959-978, 960-979, 961-980, 962-981, 963-982, 964-983, 965-984, 966-985, 967-986, 968-987, 969-988, 970-989, 971-990, 972-991, 973-992, 974-993, 975-994, 976-995, 977-996, 978-997, 979-998, 980-999, 981-1000, 982-1001, 983-1002, 984-1003, 985-1004, 986-1005, 987-1006, 988-1007, 989-1008, 990-1009, 991-1010, 992-1011, 993-1012, 994-1013, 995-1014, 996-1015, 997-1016, 998-1017, 999-1018, 1000-1019, 1001-1020, 1002-1021, 1003-1022, 1004-1023, 1005-1024, 1006-1025, 1007-1026, 1008-1027, 1009-1028, 1010-1029, 1011-1030, 1012-1031, 1013-1032, 1014-1033, 1015-1034, 1016-1035, 1017-1036, 1018-1037, 1019-1038, 1020-1039, 1021-1040,,1022-1041, 1023-1042, 1024-1043, 1025-1044, 1026-1045, 1027-1046, 1028-1047, 1029-1048, 1030-1049, 1031-1050, 1032-1051, 1033-1052, 1034-1053, 1035-1054, 1036-1055, 1037-1056, 1038-1057, 1039-1058, 1040-1059, 1041-1060, 1042-1061, 1043-1062, 1044-1063, 1045-1064, 1046-1065, 1047-1066, 1048-1067, 1049-1068, 1050-1069, 1051-1070, 1052-1071, 1053-1072, 1054-1073, 1055-1074, 1056-1075, 1057-1076, 1058-1077, 1059-1078, 1060-1079, 1061-1080, 1062-1081, 1063-1082, 1064-1083, 1065-1084, 1066-1085, 1067-1086, 1068-1087, 1069-1088, 1070-1089, 1071-1090, 1072-1091, 1073-1092, 1074-1093, 1075-1094, 1076-1095, 1077-1096, 1078-1097, 1079-1098, 1080-1099, 1081-1100, 1082-1101, 1083-1102, 1084-1103, 1085-1104, 1086-1105, 1087-1106, 1088-1107, 1089-1108, 1090-1109, 1091-1110, 1092-1111, 1093-1112, 1094-1113, 1095-1114, 1096-1115, 1097-1116, 1098-1117, 1099-1118, 1100-1119, 1101-1120, 1102-1121, 1103-1122, 1104-1123, 1105-1124, 1106-1125, 1107-1126, 1108-1127, 1109-1128, 1110-1129, 1111-1130, 1112-1131, 1113-1132, 1114-1133, 1115-1134, 1116-1135, 1117-1136, 1118-1137, 1119-1138, 1120-1139, 1121-1140, 1122-1141, 1123-1142, 1124-1143, 1125-1144, 1126-1145, 1127-1146, 1128-1147, 1129-1148, 1130-1149, 1131-1150, 1132-1151, 1133-1152, 1134-1153, 1135-1154, 1136-1155, 1137-1156, 1138-1157, 1139-1158, 1140-1159, 1141-1160, 1142-1161, 1143-1162, 1144-1163, 1145-1164, 1146-1165, 1147-1166, 1148-1167, 1149-1168, 1150-1169, 1151-1170, 1152-1171, 1153-1172, 1154-1173, 1155-1174, 1156-1175, 1157-1176, 1158-1177, 1159-1178, 1160-1179, 1161-1180, 1162-1181, 1163-1182, 1164-1183, 1165-1184, 1166-1185, 1167-1186, 1168-1187, 1169-1188, 1170-1189, 1171-1190, 1172-1191, 1173-1192, 1174-1193, 1175-1194, 1176-1195, 1177-1196, 1178-1197, 1179-1198, 1180-1199, 1181-1200, 1182-1201, 1183-1202, 1184-1203, 1185-1204, 1186-1205, 1187-1206, 1188-1207, 1189-1208, 1190-1209, 1191-1210, 1192-1211, 1193-1212, 1194-1213, 1195-1214, 1196-1215, 1197-1216, 1198-1217, 1199-1218, 1200-1219, 1201-1220, 1202-1221, 1203-1222, 1204-1223, 1205-1224, 1206-1225, 1207-1226, 1208-1227, 1209-1228, 1210-1229, 1211-1230, 1212-1231, 1213-1232, 1214-1233, 1215-1234, 1216-1235, 1217-1236, 1218-1237, 1219-1238, 1220-1239, 1221-1240, 1222-1241, 1223-1242, 1224-1243, 1225-1244, 1226-1245, 1227-1246, 1228-1247, 1229-1248, 1230-1249, 1231-1250, 1232-1251, 1233-1252, 1234-1253, 1235-1254, 1236-1255, 1237-1256, 1238-1257, 1239-1258, 1240-1259, 1241-1260, 1242-1261, 1243-1262, 1244-1263, 1245-1264, 1246-1265, 1247-1266, 1248-1267, 1249-1268, 1250-1269, 1251-1270, 1252-1271, 1253-1272, 1254-1273, 1255-1274, 1256-1275, 1257-1276, 1258-1277, 1259-1278, 1260-1279, 1261-1280, 1262-1281, 1263-1282, 1264-1283, 1265-1284, 1266-1285, 1267-1286, 1268-1287, 1269-1288, 1270-1289, 1271-1290, 1272-1291, 1273-1292, 1274-1293, 1275-1294, 1276-1295, 1277-1296, 1278-1297, 1279-1298, 1280-1299, 1281-1300, 1282-1301, 1283-1302, 1284-1303, 1285-1304, 1286-1305, 1287-1306, 1288-1307, 1289-1308, 1290-1309, 1291-1310, 1292-1311, 1293-1312, 1294-1313, 1295-1314, 1296-1315, 1297-1316; 1298-1317, 1299-1318, 1300-1319, 1301-1320, 1302-13212 1303-1322, 1304-1323, 1305-1324, 1306-1325, 1307-1326, 1308-1327, 1309-1328, 1310-1329, 1311-1330, 1312-1331, 1313-1332, 1314-1333, 1315-1334, 1316-1335, 1317-1336, 1318-1337, 1319-1338, 1320-1339, 1321-1340, 1322-1341, 1323-1342, 1324-1343, 1325-1344, 1326-1345, 1327-1346, 1328-1347, 1329-1348, 1330-1349, 1331-1350, 1332-1351, 1333-1352, 1334-1353, 1335-1354, 1336-1355, 1337-1356, 1338-1357, 1339-1358, 1340-1359, 1341-1360, 1342-1361, 1343-1362, 1344-1363, 1345-1364, 1346-1365, 1347-1366, 1348-1367, 1349-1368, 1350-1369, 1351-1370, 1352-1371, 1353-1372, 1354-1373, 1355-1374, 1356-1375, 1357-1376, 1358-1377, 1359-1378, 1360-1379, 1361-1380, 1362-1381, 1363-1382, 1364-1383, 1365-1384, 1366-1385, 1367-1386, 1368-1387, 1369-1388, 1370-1389, 1371-1390, 1372-1391, 1373-1392, 1374-1393, 1375-1394, 1376-1395, 1377-1396, 1378-1397, 1379-1398, 1380-1399, 1381-1400, 1382-1401, 1383-1402, 1384-1403, 1385-1404, 1386-1405, 1387-1406, 1388-1407, 1389-1408, 1390-1409, 1391-1410, 1392-1411, 1393-1412, 1394-1413, 1395-1414, 1396-1415, 1397-1416, 1398-1417, 1399-1418, 1400-1419, 1401-14202 1402-1421, 1403-1422, 1404-1423, 1405-1424, 1406-1425, 1407-1426, 1408-1427, 1409-1428, 1410-1429, 1411-1430, 1412-1431, 1413-1432, 1414-1433, 1415-1434, 1416-1435, 1417-1436, 1418-1437, 1419-1438, 1420-1439, 1421-1440, 1422-1441, 1423-1442, 1424-1443, 1425-1444, 1426-1445, 1427-1446, 1428-1447, 1429-1448, 1430-1449, 1431-1450, 1432-1451, 1433-1452, 1434-1453, 1435-1454, 1436-1455, 1437-1456, 1438-1457, 1439-1458, 1440-1459, 1441-1460, 1442-1461, 1443-1462, 1444-1463, 1445-1464, 1446-1465, 1447-1466, 1448-1467, 1449-1468, 1450-1469, 1451-1470, 1452-1471, 1453-1472, 1454-1473, 1455-1474, 1456-1475, 1457-1476, 1458-1477, 1459-1478, 1460-1479, 1461-1480, 1462-1481, 1463-1482, 1464-1483, 1465-1484, 1466-1485, 1467-1486, 1468-1487, 1469-1488, 1470-1489, 1471-1490, 1472-1491, 1473-1492, 1474-1493, 1475-1494, 1476-1495, 1477-1496, 1478-1497, 1479-1498, 1480-1499, 1481-1500, 1482-1501, 1483-1502, 1484-1503, 1485-1504, 1486-1505, 1487-1506, 1488-1507, 1489-1508, 1490-1509, 1491-1510, 1492-1511, 1493-1512, 1494-1513, 1495-1514, 1496-1515, 1497-1516, 1498-1517, 1499-1518, 1500-1519, 1501-1520, 1502-1521, 1503-1522, 1504-1523, 1505-1524, 1506-1525, 1507-1526, 1508-1527, 1509-1528, 1510-1529, 1511-1530, 1512-1531, 1513-1532, 1514-1533, 1515-1534, 1516-1535, 1517-1536, 1518-1537, 1519-1538, 1520-1539, 1521-1540, 1522-1541, 1523-1542, 1524-1543, 1525-1544, 1526-1545, 1527-1546, 1528-1547, 1529-1548, 1530-1549, 1531-1550, 1532-1551, 1533-1552, 1534-1553, 1535-1554, 1536-1555, 1537-1556, 1538-1557, 1539-1558, 1540-1559, 1541-1560, 1542-1561, 1543-1562, 1544-1563, 1545-1564, 1546-1565, 1547-1566, 1548-1567, 1549-1568, 1550-1569, 1551-1570, 1552-1571, 1553-1572, 1554-1573, 1555-1574, 1556-1575, 1557-1576, 1558-1577, 1559-1578, 1560-1579, 1561-1580, 1562-1581, 1563-1582, 1564-1583, 1565-1584, 1566-1585, 1567-1586, 1568-1587, 1569-1588, 1570-1589, 1571-1590, 1572-1591, 1573-1592, 1574-1593, 1575-1594, 1576-1595, 1577-1596, 1578-1597, 1579-1598, 1580-1599, and 581-1600.

Examples of 25-mer oligonucleotides capable of binding to Skp2 polynucleotides include the following oligonucleotides, indicated by polynucleotide positions with reference to SEQ ID NO:25:
1-25, 2-26, 3-27, 4-28, 5-29, 6-30, 7-31, 8-32, 9-33, 10-34, 11-35, 12-36, 13-37, 14-38, 15-39, 16-40, 17-41, 18-42, 19-43, 20-44, 21-45, 22-46, 23-47, 24-48, 25-49, 26-50, 27-51, 28-52, 29-53, 30-54, 31-55, 32-56, 33-57, 34-58, 35-59, 36-60, 37-61, 38-62, 39-63, 40-64, 41-65, 42-66, 43-67, 44-68, 45-69, 46-70, 47-71, 48-72, 49-73, 50-74, 51-75, 52-76, 53-77, 54-78, 55-79, 56-80, 57-81, 58-82, 59-83, 60-84, 61-85, 62-86, 63-87, 64-88, 65-89, 66-90, 67-91, 68-92, 69-93, 70-94, 71-95, 72-96, 73-97, 74-98, 726-309, 76-100, 77-101, 78-102, 79-103, 80-104, 81-105, 82-106, 83-107, 84-108, 85-109, 86-110, 87-111, 88-112, 89-113, 90-114, 91-115, 92-116, 93-117, 94-118, 95-119, 96-120, 97-121, 98-122, 99-123, 100-124, 101-125, 102-126, 103-127, 104-128, 105-129, 106-130, 107-131, 108-132, 109-133, 110-134, 111-135, 412-136, 113-137, 114-138, 115-139, 116-140, 117-141, 118-142, 119-143, 120-144, 121-145, 122-146, 123-147, 124-148, 125-149, 126-150, 127-151, 128-152, 129-153, 130-154, 131-155, 132-156, 133-157, 134-158, 135-159, 136-160, 137-161, 138-162, 139-163, 140-164, 141-165, 142-166, 143-167, 144-168, 145-169, 146-170, 147-171, 148-172, 149-173, 150-174, 151-175, 152-176, 153-177, 154-178, 155-179, 156-180, 157-181, 158-182, 159-183, 160-184, 161-185, 162-186, 163-187, 164-188, 165-189, 166-190, 167-191, 168-192, 169-193, 170-194, 171-195, 172-196, 173-197, 174-198, 175-199, 176-200, 177-201, 178-202, 179-203, 180-204, 181-205, 182-206, 183-207, 184-208, 185-209, 186-210, 187-211, 188-212, 189-213, 190-214, 191-215, 192-216, 193-217, 194-218, 195-219, 196-220, 197-221, 198-222, 199-223, 200-224, 201-225, 202-226, 203-227, 204-228, 205-229, 206-230, 207-231, 208-232, 209-233, 210-234, 211-235, 212-236, 213-237, 214-238, 215-239, 216-240, 217-241, 218-242, 219-243, 220-244, 221-245, 222-246, 223-247, 224-248, 225-249, 226-250, 227-251, 228-252, 229-253, 230-254, 231-255, 232-256, 233-257, 234-258, 235-259, 236-260, 237-261, 238-262, 239-263, 240-264, 241-265, 242-266, 243-267, 244-268, 245-269, 246-270, 247-271, 248-272, 249-273, 250-274, 251-275, 252-276, 253-277, 254-278, 255-279, 256-280, 257-281, 258-282, 259-283, 260-284, 261-285, 262-286, 263-287, 264-288, 265-289, 266-290, 267-291, 268-292, 269-293, 270-294, 271-295, 272-296, 273-297, 274-298, 275-299, 276-300, 277-301, 278-302, 279-303, 280-304, 281-305, 282-306, 283-307, 284-308, 285-309, 286-310, 287-311, 288-312, 289-313, 290-314, 291-315, 292-316, 293-317, 294-318, 295-319, 296-320, 297-321, 298-322, 299-323, 300-324, 301-325, 302-326, 303-327, 304-328, 305-329, 306-330, 307-331, 308-332, 309-333, 310-334, 311-335, 312-336, 313-337, 314-338, 315-339, 316-340, 317-341, 318-342, 319-343, 320-344, 321-345, 322-346, 323-347, 324-348, 325-349, 326-350, 327-351, 328-352, 329-353, 330-354, 331-355, 332-356, 333-357, 334-358, 335-359, 336-360, 337-361, 338-362, 339-363, 340-364, 341-365, 342-366, 343-367, 344-368, 345-369, 346-370, 347-371, 348-372, 349-373, 350-374, 351-375, 352-376, 353-377, 354-378, 355-379, 356-380, 357-381, 358-382, 359-383, 360-384, 361-385, 362-386, 363-387, 364-388, 365-389, 366-390, 367-391, 368-392, 369-393, 370-394, 371-395, 372-396, 373-397, 374-398, 375-399, 376-400, 377-401, 378-402, 379-403, 380-404, 381-405, 382-406, 383-407, 384-408, 385-409, 386-410, 387-411, 388-412, 389-413, 390-414, 391-415, 392-416, 393-417, 394-418, 395-419, 396-420, 397-421, 398-422, 399-423, 400-424, 401-425, 402-426, 403-427, 404-428, 405-429, 406-430, 407-431, 408-432, 409-433, 410-434, 411-435, 412-436, 413-437, 414-438, 415-439, 416-440, 417-441, 418-442, 419-443, 420-444, 421-445, 422-446, 423-447, 424-448, 425-449, 426-450, 427-451, 428-452, 429-453, 430-454, 431-455, 432-456, 433-457, 434-458, 435-459, 436-460, 437-461, 438-462, 439-463, 440-464, 441-465, 442-466, 443-467, 444-468, 445-469, 446-470, 447-471, 448-472, 449-473, 450-474, 451-475, 452-476, 453-477, 454-478, 455-479, 456-480, 457-481, 458-482, 459-483, 460-484, 461-485, 462-486, 463-487, 464-488, 465-489, 466-490, 467-491, 468-492, 469-493, 470-494, 471-495, 472-406, 473-497, 474-498, 475-499, 476-500, 477-501, 478-502, 479-503, 480-504, 481-505, 482-506, 483-507, 484-508, 485-509, 486-510, 487-511, 488-512, 489-513, 490-514, 491-515, 492-516, 493-517, 494-518, 495-519, 496-520, 497-521, 498-522, 499-523, 500-524, 501-525, 502-526, 503-527, 504-528, 505-529, 506-530, 507-531, 508-532, 509-533, 510-534, 511-535, 512-536, 513-537, 514-538, 515-539, 516-540, 517-541, 518-542, 519-543, 520-544, 521-545, 522-546, 523-547, 524-548, 525-549, 526-550, 527-551, 528-552, 529-553, 530-554, 531-555, 532-556, 533-557, 534-558, 535-559, 536-560, 537-561, 538-562, 539-563, 540-564, 541-565, 542-566, 543-567, 544-568, 545-569, 546-570, 547-571, 548-572, 549-573, 550-574, 551-575, 552-576, 553-577, 554-578, 555-579, 556-580, 557-581, 558-582, 559-583, 560-584, 561-585, 562-586, 563-587, 564-588, 565-589, 566-590, 567-591, 568-592, 569-593, 570-594, 571-595, 572-596, 573-597, 574-598, 575-599, 576-600, 577-601, 578-602, 579-603, 580-604, 581-605, 582-606, 583-607, 584-608, 585-609, 586-610, 587-611, 588-612, 589-613, 590-614, 591-615, 592-616, 593-617, 594-618, 595-619, 596-620, 597-621, 598-622, 599-623, 600-624, 601-625, 602-626, 603-627, 604-628, 605-629, 606-630, 607-631, 608-632, 609-633, 610-634, 611-635, 612-636, 613-637, 614-638, 615-639, 621-645, 622-646, 623-647, 624-648, 625-649, 626-650, 627-651, 628-652, 629-653, 630-654, 631-655, 632-656, 633-657, 634-658, 635-659, 636-660, 637-661, 638-662, 639-663, 640-664, 641-665, 642-666, 643-667, 644-668, 645-669, 646-670, 647-671, 648-672, 649-673, 650-674, 651-675, 652-676, 653-677, 654-678, 655-679, 656-680, 657-681, 658-682, 659-683, 660-684, 661-685, 662-686, 663-687, 664-688, 665-689, 666-690, 667-691, 668-692, 669-693, 670-694, 671-695, 672-696, 673-697, 674-698, 675-699, 676-700, 677-701, 678-702, 679-703, 680-704, 681-705, 682-706, 683-707, 684-708, 685-709, 686-710, 687-711, 688-712, 689-713, 690-714, 691-715, 692-716, 693-717, 694-718, 695-719, 696-720, 697-721, 698-722, 699-723, 700-724, 701-725, 702-726, 703-727, 704-728, 705-729, 706-730, 707-731, 708-732, 709-733, 710-734, 711-735, 712-736, 713-737, 714-738, 715-739, 716-740, 717-741, 718-742, 719-743, 720-744, 721-745, 722-746, 723-747, 724-748, 725-749, 726-750, 727-751, 728-752, 729-753, 730-754, 731-755, 732-756, 733-757, 734-758, 735-759, 736-760, 737-761, 738-762, 739-763, 740-764, 741-765, 742-766, 743-767, 144-768, 745-769, 746-770, 747-771, 748-772, 749-773, 750-774, 751-775, 752-176, 753-777, 754-778, 755-779, 756-780, 757-781, 758-782, 759-783, 760-784, 761-785, 762-786, 763-787, 764-788, 765-789, 766-790, 767-791, 768-792, 769-793, 770-794, 771-795, 772-796, 773-797, 774-798, 775-799, 776-800, 777-801, 778-802, 779-803, 780-804, 781-805, 782-806, 783-807, 784-808, 785-809, 786-810, 787-811, 788-812, 789-813, 790-814, 791-815, 792-816, 793-817, 794-818, 795-819, 796-820, 797-821, 798-822, 799-823, 800-824, 801-825, 802-826, 803-827, 804-828, 805-829, 806-830, 807-831, 808-832, 809-833, 810-834, 811-835, 812-836, 813-837, 814-838, 815-839, 816-840, 817-841, 818-842, 819-843, 820-844, 821-845, 822-846, 823-847, 824-848, 825-849, 826-850, 827-851, 828-852, 829-853, 830-854, 831-855, 832-856, 833-857, 834-858, 835-859, 836-860, 837-861, 838-862, 839-863, 840-864, 841-865, 842-866, 843-867, 844-868, 845-869, 846-870, 847-871, 848-872, 849-873, 850-874, 851-875, 852-876, 853-877, 854-878, 855-879, 856-880, 857-881, 858-882, 859-883, 860-884, 861-885, 862-886, 863-887, 864-888, 865-889, 866-890, 867-891, 868-892, 869-893, 870-894, 871-895, 872-896, 873-897, 874-898, 875-899, 876-900, 877-901, 878-902, 879-903, 880-904, 881-905, 882-906, 883-907, 884-908, 885-909, 886-910, 887-911, 888-912, 889-913, 890-914, 891-915, 892-916, 893-917, 894-918, 895-919, 896-920, 897-921, 898-922, 899-923, 900-924, 901-925, 902-926, 903-927, 904-928, 905-929, 906-930, 907-931, 908-932, 909-933, 910-934, 911-935, 912-936, 913-937, 914-938, 915-939, 916-940, 917-941, 918-942, 919-943, 920-944, 921-945, 922-946, 923-947, 924-948, 925-949, 926-950, 927-951, 928-952, 929-953, 930-954, 931-955, 932-956, 933-957, 934-958, 935-959, 936-960, 937-961, 938-962, 939-963, 940-964, 941-965, 942-966, 943-967, 944-968, 945-969, 946-970, 947-971, 948-972, 949-973, 950-974, 951-975, 952-976, 953-977, 954-978, 955-979, 956-980, 957-981, 958-982, 959-983, 960-984, 961-985, 962-986, 963-987, 964-988, 965-989, 966-990, 967-991, 968-992, 969-993, 970-994, 971-995, 972-996, 973-997, 974-998, 975-999, 976-1000, 977-1001, 978-1002, 979-1003, 980-1004, 981-1005, 982-1006, 983-1007, 984-1008, 985-1009, 986-1010, 987-1011, 988-1012, 989-1013, 990-1014, 991-1015, 992-1016, 993-1017, 994-1018, 995-1019, 996-1020, 997-1021, 998-1022, 999-1023, 1000-1024, 1001-1025, 1002-1026, 1003-1027, 1004-1028, 1005-1029, 1006-1030, 1007-1031, 1008-1032, 1009-1033, 1010-1034, 1011-1035, 1012-1036, 1013-1037, 1014-1038, 1015-1039, 1016-1040, 1017-1041, 1018-1042, 1019-1043, 1020-1044, 1021-1045, 1022-1046, 1023-1047, 1024-1048, 1025-1049, 1026-1050, 1027-1051, 1028-1052, 1029-1053, 1030-1054, 1031-1055, 1032-1056, 1033-1057, 1034-1058, 1035-1059, 1036-1060, 1037-1061, 1038-1062, 1039-1063, 1040-1064, 1041-1065, 1042-1066, 1043-1067, 1044-1068, 1045-1069, 1046-1070, 1047-1071, 1048-1072, 1049-1073, 1050-1074, 1051-1075, 1052-1076, 1053-1077, 1054-1078, 1055-1079, 1056-1080, 1057-1081, 1058-1082, 1059-1083, 1060-1084, 1061-1085, 1062-1086, 1063-1087, 1064-1088, 1065-1089, 1066-1090, 1067-1091, 1068-1092, 1069-1093, 1070-1094, 1071-1095, 1072-1096, 1073-1097, 1074-1098, 1075-1099, 1076-1100, 1077-1101, 1078-1102, 1079-1103, 1080-1104, 1081-1105, 1082-1106, 1083-1107, 1084-1108, 1085-1109, 1086-1110, 1087-1111, 1088-1112, 1089-1113, 1090-1114, 1091-1115, 1092-1116, 1093-1117, 1094-1118, 1095-1119, 1096-1120, 1097-1121, 1098-1122, 1099-1123, 1100-1124, 1101-1125, 1102-1126, 1103-1127, 1104-1128, 1105-1129, 1106-1130, 1107-1131, 1108-1132, 1109-1133, 1110-1134, 1111-1135, 1112-1136, 1113-1137, 1114-1138, 1115-1139, 1116-1140, 1117-1141, 1118-1142, 1119-1143, 1120-1144, 1121-1145, 1122-1146, 1123-1147, 1124-1148, 1125-1149, 1126-1150, 1127-1151, 1128-1152, 1129-1153, 1130-1154, 1131-1155, 1132-1156, 1133-1157, 1134-1158, 1135-1159, 1136-1160, 1137-1161, 1138-1162, 1139-1163, 1140-1164, 1141-1165, 1142-1166, 1143-1167, 1144-1168, 1145-1169, 1146-1170, 1147-1171, 1148-1172, 1149-1173, 1150-1174, 1151-1175, 1152-1176, 1153-1177, 1154-1178, 1155-1179, 1156-1180, 1157-1181, 1158-1182, 1159-1183, 1160-1184, 1161-1185, 1162-1186, 1163-1187, 1164-1188, 1165-1189, 1166-1190, 1167-1191, 1168-1192, 1169-1193, 1170-1194, 1171-1195, 1172-1196, 1173-1197, 1174-1198, 1175-1199, 1176-1200, 1177-1201, 1178-1202, 1179-1203, 1180-1204, 1181-1205, 1182-1206, 1183-1207, 1184-1208, 1185-1209, 1186-1210, 1187-1211, 1188-1212, 1189-1213, 1190-1214, 1191-1215, 1192-1216, 1193-1217, 1194-1218, 1195-1219, 1196-1220, 1197-1221, 1198-1222, 1199-1223, 1200-1224, 1201-1225, 1202-1226, 1203-1227, 1204-1228, 1205-1229, 1206-1230, 1207-1231, 1208-1232, 1209-1233, 1210-1234, 1211-1235, 1212-1236, 1213-1237, 1214-1238, 1215-1239, 1216-1240, 1217-1241, 1218-1242, 1219-1243, 1220-1244, 1221-1245, 1222-1246, 1223-1247, 1224-1248, 1225-1249, 1226-1250, 1227-1251, 1228-1252, 1229-1253, 1230-1254, 1231-1255, 1232-1256, 1233-1257, 1234-1258, 1235-1259, 1236-1260, 1237-1261, 1238-1202, 1239-1263, 1240-1264, 1241-1265, 1242-1266, 1243-1267, 1244-1268, 1245-1269, 1246-1270, 1247-1271, 1248-1272, 1249-1273, 1250-1274, 1251-1275, 1252-1276, 1253-1277, 1254-1278, 1255-1279, 1256-1280, 1257-1281, 1258-1282, 1259-1283, 1260-1284, 1261-1285, 1262-1286, 1263-1287, 1264-1288, 1265-1289, 1266-1290, 1267-1291, 1268-1292, 1269-1293, 1270-1294, 1271-1295, 1272-1296, 1273-1297, 1274-1298, 1275-1299, 1276-1300, 1277-1301, 1278-1302, 1279-1303, 1280-1304, 1281-1305, 1282-1306, 1283-1307, 1284-1308, 1285-1309, 1286-1310, 1287-1311, 1288-1312, 1289-1313, 1290-1314, 1291-1315, 1292-1316, 1293-1317, 1294-1318, 1295-1319, 1296-1320, 1297-1321, 1298-1322, 1299-1323, 1300-1324, 1301-1325, 1302-1326, 1303-1327, 1304-1328, 1305-1329, 1306-1330, 1307-1331, 1308-1332, 1309-1333, 1310-1334, 1311-1335, 1312-1336, 1313-1337, 1314-1338, 1315-1339, 1316-1340, 1317-1341, 1318-1342, 1319-1343, 1320-1344, 1321-1345, 1322-1346, 1323-1347, 1324-1348, 1325-1349, 1326-1350, 1327-1351, 1328-1352, 1329-1353, 1330-1354, 1331-1355, 1332-1356, 1333-1357, 1334-1358, 1335-1359, 1336-1360, 1337-1361, 1338-1362, 1339-1363, 1340-1364, 1341-1365, 1342-1366, 1343-1367, 1344-1368, 1345-1369, 1346-1370, 1347-1371, 1348-1372, 1349-1373, 1350-1374, 1351-1375, 1352-1376, 1353-1377, 1354-1378, 1355-1379, 1356-1380, 1357-1381, 1358-1382, 1359-1383, 1360-1384, 1361-1385, 1362-1386, 1363-1387, 1364-1388, 1365-1389, 1366-1390, 1367-1391, 1368-1392, 1369-1393, 1370-1394, 1371-1395, 1372-1396, 1373-1397, 1374-1398, 1375-1399, 1376-1400, 1377-1401, 1378-1402, 1379-1403, 1380-1404, 1381-1405, 1382-1406, 1383-1407, 1384-1408, 1385-1409, 1386-1410, 1387-1411, 1388-1412, 1389-1413, 1390-1414, 1391-1415, 1392-1416, 1393-1417, 1394-1418, 1395-1419, 1396-1420, 1397-1421, 1398-1422, 1399-1423, 1400-1424, 1401-1425, 1402-1426, 1403-1427, 1404-1428, 1405-1429, 1406-1430, 1407-1431, 1408-1432, 1409-1433, 1410-1434, 1411-1435, 1412-1436, 1413-1437, 1414-1438, 1415-1439, 1416-1440, 1417-1441, 1418-1442, 1419-1443, 1420-1444, 1421-1445, 1422-1446, 1423-1447, 1424-1448, 1425-1449, 1426-1450, 1427-1451, 1428-1452, 1429-1453, 1430-1454, 1431-1455, 1432-1456, 1433-1457, 1434-1458, 1435-1459, 1436-1460, 1437-1461, 1438-1462, 1439-1463, 1440-1464, 1441-1465, 1442-1466, 1443-1467, 1444-1468, 1445-1469, 1446-1470, 1447-1471, 1448-1412, 1449-1473, 1450-1474, 1451-1475, 1452-1476, 1453-1477, 1454-1478, 1455-1479, 1456-1480, 1457-1481, 1458-1482, 1459-1483, 1460-1484, 1461-1485, 1462-1486, 1463-1487, 1464-1488, 1465-1489, 1466-1490, 1467-1491, 1468-1492, 1469-1493, 1470-1494, 1471-1495, 1472-1496, 1473-1497, 1474-1498, 1475-1499, 1476-1500, 1477-156;1 1478-1502, 1479-1503, 1480-1504, 1481-1505, 1482-1506, 1483-1507, 1484-1508, 1485-1509, 1486-1510, 1487-1511, 1488-1512, 1489-1513, 1490-1514, 1491-1515, 1492-1516, 1493-1517, 1494-1518, 1495-1519, 1496-1520, 1497-1521, 1498-1522, 1499-1523, 1500-1524, 1501-1525, 1502-1526, 1503-1527, 1504-1528, 1505-1529, 1506-1530, 1507-1531, 1508-1532, 1509-1533, 1510-1534, 1511-1535, 1512-1536, 1513-1537, 1514-1538, 1515-1539, 1516-1540, 1517-1541, 1518-1542, 1519-1543, 1520-1544, 1521-1545, 1522-1546, 1523-1547, 1524-1548, 1525-1549, 1526-1550, 1527-1551, 1528-1552, 1529-1553, 1530-1554, 1531-1555, 1532-1556, 1533-1557, 1534-1558, 1535-1559, 1536-1560, 1537-1561, 1538-1562, 1539-1563, 1540-1564, 1541-1565, 1542-1566, 1543-1567, 1544-1568, 1545-1569, 1546-1570, 1547-1571, 1548-1572, 1549-1573, 1550-1574, 1551-1575, 1552-1576, 1553-1577, 1554-1578, 1555-1579, 1556-1580, 1557-1581, 1558-1582, 1559-1583, 1560-1584, 1561-1585, 1562-1586, 1563-1587, 1564-1588, 1565-1589, 1566-1590, 1567-1591, 1568-1592, 1569-1593, 1570-1594, 1571-1595, 1572-1596, 1573-1597, 1574-1598, 1575-1599, 1576-1600.

Preferred Skp2 antisense oligonucleotides include:

| | |
|---|---|
| skp2-1 AS | |
| AGACAGTATGCCGTGGAGGGTGGAC | (SEQ ID NO:5) |
| skp2-2 AS | |
| CTTCGCCTTCCAGATTCCCGCTTTG | (SEQ ID NO:7) |
| skp2-3 AS | |
| CCCCTTGAGACAGCAACCGACCAGT | (SEQ ID NO:9) |
| skp2-4 AS | |
| AGGACACCCAGGAAGGTTAAGTCGC | (SEQ ID NO:11) |
| skp2-5 AS | |
| GGCAATGGTGGTGAAATGGGAGCAA | (SEQ ID NO:13) |
| skp2-6 AS | |
| GTTCTCACTGTCGGGCTCCTCTTTC | (SEQ ID NO:15) |
| skp2-7 AS | |
| GGCAATCACCCCTTGAGACAGCAAC | (SEQ ID NO:17) |
| skp2-8 AS | |
| TGAGACAGTATGCCGTGGAGGGTGG | (SEQ ID NO:19) |
| skp2-9 AS | |
| TGGGCTTTTGCAGTGTCAGTCGGC | (SEQ ID NO:21) |
| skp2-10 AS | |
| CCCCATGCCTGACAGCAGTTCAGAA | (SEQ ID NO:23) |

The present invention relates to antisense oligonucleotides designed to interfere with the normal function of Cks1 or Skp2 polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in U.S. Pat. No. 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 25 5,565,350, 5,652,355, 5,700,922 and 5,958,773.

Preferred antisense oligonucleotides in addition to those of SEQ ID NO:26-30 can be selected by routine experimentation using, for example, assays described in the Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles, R. V. et al., Nuc. Acids Res. (1995) 23:954-961; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

As disclosed in U.S. Pat. No. 5,998,383, incorporated herein by reference, the oligonucleotide is selected such that the sequence exhibits suitable energy related characteristics important for oligonucleotide duplex formation with their complementary templates, and shows a low potential for self-dimerization or self-complementation (Anazodo et al., Biochem. Biophys. Res. Commun. (1996) 229:305-309). The computer program OLIGO (Primer Analysis Software, Version 3.4), is used to determined antisense sequence melting temperature, free energy properties, and to estimate potential self-dimer formation and self-complimentarity properties. The program allows the determination of a qualitative estimation of these two parameters (potential self-dimer formation and self-complimentary) and provides an indication of “no potential” or “some potential” or “essentially complete potential.” Segments of Cks1 polynucleotides are generally selected that have estimates of no potential in these parameters. However, segments can be used that have “some potential” in one of the categories. A balance of the parameters is used in the selection.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in Example 1. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. (1998) 23;45-50.) According to the present invention, cultures of SW620 cells were transfected with five different antisense oligonucleotides designed to target Cks1. These oligonucleotides are shown in SEQ ID NO:26-30. The levels of mRNA corresponding to Cks1 were measured in treated and control cells. SEQ ID NO:26-30 caused dramatic decreases in Cks1 mRNA when normalized to actin mRNA levels.

Additional inhibitors include ribozymes, proteins or polypeptides, antibodies or fragments thereof as well as small molecules. Each of these Cks1 and Skp2 inhibitors share the common feature in that they reduce the expression and/or biological activity of Cks1 and Skp2, respectively. In addition to the exemplary Cks1 and Skp2 inhibitors disclosed herein, alternative inhibitors may be obtained through routine experimentation utilizing methodology either specifically disclosed herein or as otherwise readily available to and within the expertise of the skilled artisan.

Ribozymes

Cks1 and Skp2 inhibitors may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell (1987) 48:211-220; Haseloff and Gerlach, Nature (1988) 328:596-600; Walbot and Bruening, Nature (1988) 334:196; Haseloff and Gerlach, Nature (1988) 334:585); the hairpin ribozyme (for example, as described by Haseloffet al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such Cks1 mRNA-specific ribozyme, or a nucleic acid encoding such a ribozyme, may be delivered to a host cell to effect inhibition of Cks1 gene expression. Ribozymes and the like may therefore be delivered to the host cells by DNA encoding the ribozyme linked to a eukaryotic promoter, such as a eukaryolic viral promoter, such that upon introduction into the nucleus, the ribozyme will be directly transcribed.

Proteins and Polypeptides

In addition to the antisense molecules and ribozymes disclosed herein, Cks1 inhibitors of the present invention also include proteins or polypeptides that are effective in either reducing Cks1 gene expression or in decreasing one or more of Cks1's biological activities. A variety of methods are readily available in the art by which the skilled artisan may, through routine experimentation, rapidly identify such Cks1 inhibitors. The present invention is not limited by the following exemplary methodologies.

Inhibitors of Cks1 biological activities and Skp2 biological activities encompass those proteins and/or polypeptides that interfere with Cks1 or Skp2 activity. Such interference may occur through direct interaction with Cks1 or Skp2 active domain or indirectly through non- or un-competitive inhibition such as via binding to an allosteric site. Accordingly, available methods for identifying proteins and/or polypeptides that bind to Cks1 or Skp2 may be employed to identify lead compounds that may, through the methodology disclosed herein, be characterized for their Cks1 or Skp2 inhibitory activity.

Literature is available to the skilled artisan that describes methods for detecting and analyzing protein-protein interactions. Reviewed in Phizicky, E. M. et al., Microbiological Reviews (1995) 59:94-123 incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive Cks1 or Skp2 inhibitors may be identified through biological screening assays that rely on the direct interaction between the Cks1 or Skp2 protein and a panel or library of potential inhibitor proteins. Biological screening methodologies, including the various “n-hybrid technologies,” are described in, for example, Vidal, M. et al., Nucl. Acids Res. (1999) 27(4):919-929; Frederickson, R. M., Curr Opin. Biotechnol. (1998) 9(1):90-6; Brachmann, R. K. et al., Curr. Opin. Biotechnol. (1997) 8(5):561-568; and White, M. A., Proc. Natl. Acad. Sci. U.S.A. (1996) 93:10001-10003 each of which is incorporated herein by reference.

The two-hybrid screening methodology may be employed to search new or existing target cDNA libraries for Cks1 or Skp2 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C.-T. et al., Proc. Natl. Acad. Sci. U.S.A. (1991) 88:9578-9582; Dalton, S. et al., Cell (1992) 68:597-612; Durfee, T. K. et al., Genes Pev. (1993) 7:555-569; Vojtek, A. B. et al., Cell (1993) 74:205-214; and Zervos, A. S. et al., Cell (1993) 72:223-232. Commonly used reporter genes include the E. coli lacZ gene as well as selectable yeast genes such as HIS3 and LEU2. Fields, S. et al., Nature (London) (1989) 340:245-246; Durfee, T. K., supra; and Zervos, A. S., supra. A wide variety of activation domain libraries is readily available in the art such that the screening for interacting proteins may be performed through routine experimentation.

Suitable bait proteins for the identification of Cks1 and Skp2 interacting proteins may be designed based on the Cks1 or Skp2 cDNA sequence presented herein as SEQ ID NO:1 and SEQ ID NO:3, respectively. Such bait proteins include either the full-length Cks1 protein or fragments thereof.

Plasmid vectors, such as, e.g., pBTM116 and pAS2-1, for preparing Cks1 and Skp2 bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

Cks1 or Skp2 inhibitors of the present invention may alternatively be identified through one of the physical or biochemical methods available in the art for detecting protein-protein interactions.

Through the protein affinity chromatography methodology, lead compounds to be tested as potential Cks1 or Skp2 inhibitors may be identified by virtue of their specific retention to Cks1 or Skp2 when either covalently or non-covalently coupled to a solid matrix such as, e.g., Sepharose beads. The preparation of protein affinity columns is described in, for example, Beeckmans, S. et al., Eur. J. Biochem. (1981) 117:527-535 and Formosa, T. et al., Methods Enzymol. (1991) 208:24-45. Cell lysates containing the full complement of cellular proteins may be passed through the Cks1 or Skp2 affinity column. Proteins having a high affinity for Cks1 or Skp2 will be specifically retained under low-salt conditions while the majority of cellular proteins will pass through the column. Such high affinity proteins may be eluted from the immobilized Cks1 or Skp2 under conditions of high-salt, with chaotropic solvents or with sodium dodecyl sulfate (SDS). In some embodiments, it may be preferred to radiolabel the cells prior to preparing the lysate as an aid in identifying the Cks1 or Skp2 specific binding proteins. Methods for radiolabeling mammalian cells are well known in the art and are provided, e.g., in Sopta, M. et al., J. Biol. Chem. (1985) 260:10353-10360.

Suitable Cks1 or Skp2 proteins for affinity chromatography may be fused to a protein or polypeptide to permit rapid purification on an appropriate affinity resin. For example, the Cks1 or Skp2 cDNA may be fused to the coding region for glutathione S-transferase (GST) which facilitates the adsorption of fusion proteins to glutathione-agarose columns. Smith et al., Gene (1988) 67:31-40. Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of Cks1 or Skp2 fusion proteins that is presented herein is the epitope for the influenza virus hemagglutinin (HA) against which monoclonal antibodies are readily available and from which antibodies an affinity column may be prepared.

Proteins that are specifically retained on a Cks1 or Skp2 affinity column may be identified after subjecting to SDS polyacrylamide gel electrophoresis (SDS-PAGE). Thus, where cells are radiolabeled prior to the preparation of cell lysates and passage through the Cks1 or Skp2 affinity column, proteins having high affinity for Cks1 or Skp2 may be detected by autoradiography. The identity of Cks1 or Skp2 specific binding proteins may be determined by protein sequencing techniques that are readily available to the skilled artisan, such as Mathews, C. K. et al., Biochemistry, The Benjamin/Cummings Publishing Company, Inc. pp.166-170 (1990).

Antibodies or Antibody Fragments

Cks1 or Skp2 inhibitors of the present invention include antibodies and/or antibody fragments that are effective in reducing Cks1 or Skp2 gene expression and/or biological activity. Suitable antibodies may be monoclonal, polyclonal or humanized monoclonal antibodies. Antibodies may be derived by conventional hybridoma based methodology, from antisera isolated from or Cks1 or Skp2 inoculated animals or through recombinant DNA technology. Alternatively, inventive antibodies or antibody fragments may be identified in vitro by use of one or more of the readily available phage display libraries. Exemplary methods are disclosed herein.

In one embodiment of the present invention, Cks1 or Skp2 inhibitors are monoclonal antibodies that maybe produced as follows. Cks1 or Skp2 protein may be produced, for example, by expression of Cks1 or Skp2 cDNA in a baculovirus based system. By this method, Cks1 or Skp2 cDNA or a fragment thereof is ligated into a suitable plasmid vector that is subsequently used to transfect Sf9 cells to facilitate protein production. In addition, it may be advantageous to incorporate an epitope tag or other moiety to facilitate affinity purification of the Cks1 or Skp2 protein. Clones of Sf9 cells expressing Cks1 or Skp2 are identified, e.g., by enzyme linked immunosorbant assay (ELISA), lysates are prepared and the Cks1 or Skp2 protein purified by affinity chromatography and the purified protein is injected, intraperitoneally, into BALB/c mice to induce antibody production. It may be advantageous to add an adjuvant, such as Freund's adjuvant, to increase the resulting immune response.

Serum is tested for the production of specific antibodies and spleen cells from animals having a positive specific antibody titer are used for cell fusions with myeloma cells to generate hybridoma clones. Supernatants derived from hybridoma clones are tested for the presence of monoclonal antibodies having specificity against Cks1 or Skp2. For a general description of monoclonal antibody methodology, See, e.g., Harlow and Lane, Antibodies: A Laboratory Manual, Cold Spring Harbor Laboratory (1988).

In addition to the baculovirus expression system, other suitable bacterial or yeast expression systems may be employed for the expression of Cks1 or Skp2 protein or polypeptides thereof. As with the baculovirus system, it may be advantageous to utilize one of the commercially available affinity tags to facilitate purification prior to inoculation of the animals. Thus, the Cks1 or Skp2 cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In other embodiments of the present invention, Cks1 or Skp2 inhibitors are humanized anti-Cks1 or Skp2 monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature (1986) 321:522-525; Morrison et al., Proc. Natl. Acad. Sci., U.S.A., (1984) 81:6851-6855; Morrison and Oi, Adv. Immunol. (1988) 44:626-302; Verhoeyer et al., Science (1988) 239:1534-1536; Padlan, Molec. Immun. (1991) 28:489-498; Padlan, Molec. Immunol. (1994) 31(3):169-217; and Kettleborough, C. A. et al., Protein Eng. (1991) 4:773-83 each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. (1987) 196:901-917; Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Humanized antibodies to Cks1 or Skp2 can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses- methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFa, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, Cks1 or Skp2 polypeptides of the invention and variants thereof are used to immunize a transgenic animal as described above. Monoclonal antibodies are made using methods known in the art, and the specificity of the antibodies is tested using isolated Cks1 or Skp2 polypeptides. The suitability of the antibodies for clinical use is tested by, for example, exposing SW620 cells to the antibodies and measuring cell growth. According to the invention, inhibition of or Cks1 or Skp2 expression using antisense oligonucleotides specific for Cks1 or Skp2 polynucleotides causes an inhibition of anchorage-independent growth of a colon cancer cell line, SW620. The antisense oligonucleotides also inhibited the proliferation of a ovarian cancer cell line, SKOV3. Human monoclonal antibodies specific for Cks1 or Skp2 or a variant or fragment thereof can be tested for their ability to inhibit proliferation, colony growth, or any other biological parameter indicative of control of tumor growth, migration, or metastasis, particularly tumor cells of epithelial origin. Such antibodies would be suitable for pre-clinical and clinical trials as pharmaceutical agents for preventing or controlling growth of cancer cells.

It will be appreciated that alternative Cks1 or Skp2 inhibitor antibodies may be readily obtained by other methods commonly known in the art. One exemplary methodology for identifying antibodies having a high specificity for Cks1 is the phage display technology.

Phage display libraries for the production of high-affinity antibodies are described in, for example, Hoogenboom, H. R. et al., Immunotechnology (1998) 4(1):1-20, Hogenboom, H. R., Trends Biotechnol. (1997) 15:62-70 and McGuinness B. et al., Nature Bio. Technol. (1996) 14:1149-1154 each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature (1990) 348:552-554 which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

Cks1 or Skp2 protein suitable for screening a phage library may be obtained by, for example, expression in baculovirus Sf9 cells as described, supra. Alternatively, the Cks1 or Skp2 coding region may be PCR amplified using primers specific to the desired region of the Cks1 or Skp2 protein. As discussed above, the Cks1 protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-Cks1 or Skp2 binding properties may subsequently be isolated by successive panning, in the case of a solid matrix, or by affinity adsorption to a Cks1 or Skp2 antigen column. Phage having the desired Cks1 inhibitory activities may be reintroduced into bacteria by infection and propagated by standard methods known to those skilled in the art See Hoogenboom, H. R., Trends Biotechnol., supra for a review of methods for screening for positive antibody-pIII phage.

Small Molecules

The present invention also provides small molecule Cks1 or Skp2 inhibitors that may be readily identified through routine application of high-throughput screening (HTS) methodologies. Reviewed by Persidis, A., Nature Biotechnology (1998) 16:488-489. HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. See, e.g., Gonzalez, J. E. et al., Curr. Opin. Biotechnol. (1998) 9(6):624-631 incorporated herein by reference.

HTS methodology may be employed, e.g., to screen for lead compounds that block one of Cks1's or Skp2's biological activities. By this method, Cks1 or Skp2 protein may be immunoprecipitated from cells expressing the protein and applied to wells on an assay plate suitable for robotic screening. Individual test compounds may then be contacted with the immunoprecipitated protein and the effect of each test compound on Cks1 or Skp2 kinase activity assessed by, e.g., incubating in the presence of γ-³²P-ATP in a suitable buffer system, and measuring the incorporation of ³²P.

Methods for Assessing the Efficacy of Cks1 and Skp2 Inhibitors

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit Cks1 or Skp2 gene expressor or biological activity. As discussed in further detail in the Examples provided below, Cks1 or Skp2 inhibitors of the present invention are effective in reducing Cks1 or Skp2 expression levels. Thus, the present invention further discloses methods that permit the skilled artisan to assess the effect of candidate inhibitors.

Candidate Cks1 or Skp2 inhibitors may be tested by administration to cells that either express endogenous Cks1 or Skp2 or that are made to express Cks1 or Skp2 by transfection of a mammalian cell with recombinant Cks1 or Skp2 plasmid construct.

Effective Cks1 or Skp2 inhibitory molecules will be effective in reducing the levels of Cks1 or Skp2 mRNA as determined, e.g., by Northern blot or RT-PCR analysis. For a general description of these procedures, see, e.g., Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pasternak, J. J. 1998) incorporated herein by reference. The effectiveness of a given candidate antisense molecule may be assessed by comparison with a control “antisense” molecule known to have no substantial effect on Cks1 or Skp2 expression when administered to a mammalian cell. Exemplary control molecules include the Cks1 or Skp2 oligonucleotides disclosed as SEQ ID NO:31-35 (Cks1) and SEQ ID NO:6, 8, 10, 12, 14, 16, 18, 20, 22 and 24 (Skp2).

In alternate embodiments of the present invention, the effect of Cks1 or Skp2 inhibitors on the rate of DNA synthesis after challenge with a radiation or chemotherapeutic agent may be assessed by, e.g., the method of Young and Painter. Hum. Genet. (1989) 82:113-117. Briefly, culture cells may be incubated in the presence of ¹⁴C-thymidine prior to exposure to, e.g., X-rays. Immediately after irradiation, cells are incubated for a short period prior to addition of ³H-thymidine. Cells are washed, treated with perchloric acid and filtered (Whatman GF/C). The filters are rinsed with perchloric acid, 70% alcohol and then 100% ethanol; radioactivity is measured and the resulting ³H/¹⁴C ratios used to determine the rates of DNA synthesis.

Cks1 or Skp2 inhibitors effective in reducing Cks1 or Skp2 gene expression by one or more of the methods discussed above may be further characterized in vivo for efficacy in one of the readily available animal model systems. Various animal model systems for study of cancer and genetic instability associated genes are disclosed in, for example, Donehower, L. A. Cancer Surveys (1997) 29:329-352 incorporated herein by reference.

Pharmaceutical Compositions

The antisense oligonucleotides and ribozymes of the present invention can be synthesized by any method known in the art for ribonucleic or deoxyribonucleic nucleotides. For example, the oligonucleotides can be prepared using solid-phase, synthesis such as in an Applied Biosystems 380B DNA synthesizer. Final purity of the oligonucleotides is determined as is known in the art.

The antisense oligonucleotides identified using the methods of the invention modulate tumor cell proliferation. Therefore, pharmaceutical compositions and methods are provided for interfering with cell proliferation, preferably tumor cell proliferation, comprising contacting tissues or cells with one or more of antisense oligonucleotides identified using the methods of the invention. Preferably, an antisense oligonucleotide having one of SEQ ID NOS:26-30 or SEQ ID NO:5, 7, 9, 11, 13, 15, 17, 19, 21 and 23 is administered.

The methods and compositions may also be used to treat proliferative disorders including other forms of cancer such as leukemias, lymphomas (Hodgkins and non-Hodgkins), sarcomas, melanomas, adenomas, carcinomas of solid tissue, hypoxic tumors, squamous cell carcinomas of the mouth, throat, larynx, and lung, genitourinary cancers such as cervical and bladder cancer, hematopoietic cancers, colon cancer, pancreatic cancer, head and neck cancers, and nervous system cancers, benign lesions such as papillomas, arthrosclerosis, psoriasis, primary and secondary polythemia, mastocytosis, autoimmune diseases, angiogenesis, bacterial infections, and viral infections, such as HIV infections, hepatitis or herpes infections.

The invention provides pharmaceutical compositions of antisense oligonucleotides and ribozymes complementary to the Cks1 or Skp2 mRNA gene sequence as active ingredients for therapeutic application. These compositions can also be used in the method of the present invention. Where required the compounds are nuclease resistant. In general the pharmaceutical composition for modulating cell proliferation or for cytotoxicity in a mammal includes an effective amount of at least one antisense oligonucleotide as described above needed for the practice of the invention, or a fragment thereof shown to have the same effect, and a pharmaceutically physiologically acceptable carrier or diluent.

In one embodiment of the invention, a method is provided for reducing metastasis in a subject comprising administering an amount of an antisense oligonucleotide of the invention effective to reduce metastasis. Most preferably the antisense oligonucleotide is one of SEQ ID NOS:26-30.

The pharmaceutical composition for inhibiting tumorigenicity of neoplastic cells in a mammal consists of an effective amount of at least one active ingredient selected from antisense oligonucleotides complementary to the Cks1 or Skp2 mRNA, including the entire Cks1 or Skp2 mRNA or having short sequences as set forth in SEQ ID NOS:26-30 (Cks1) or SEQ ID NO:5, 7, 9, 11, 13, 15, 17, 19, 21 and 23 (Skp2) and a pharmaceutically physiologically acceptable carrier or diluent. Combinations of the active ingredients can be used.

The compositions can be administered orally, subcutaneously or parenterally including intravenous, intraarterial, intramuscular, intraperitoneally, and intranasal administration as well as intrathecal and infusion techniques as required by the malignant cells being treated. For delivery within the CNS intrathecal delivery can be used with for example an Ommaya reservoir or other methods known in the art. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate oligonucleotide uptake. Implants of the compounds are also useful. In general, the pharmaceutical compositions are sterile.

In the method of the present invention, proliferating cells including neoplastic cells are contacted with a growth-inhibiting amount of the bioactive antisense oligonucleotide for the Cks1 or Skp2 mRNA or a fragment thereof shown to have substantially the same effect. In an embodiment, the mammal to be treated is human but other mammalian species can be treated in veterinary applications.

By bioactive (expressible) is meant that the oligonucleotide is biologically active in the cell when delivered directly to the cell and/or is expressed by an appropriate promotor and active when delivered to the cell in a vector as described below. Nuclease resistance is provided by any method known in the art that does not substantially interfere with biological activity as described herein.

“Contacting the cell” refers to methods of exposing or delivery to a cell of antisense oligonucleotides whether directly or by viral or non-viral vectors and where the antisense oligonucleotide is bioactive upon delivery. The method of delivery will be chosen for the particular cancer being treated. Parameters that affect delivery can include the cell type affected and tumor location as is known in the medical art.

The treatment generally has a length proportional to the length of the disease process and drug effectiveness and the patient species being treated. It is noted that humans are treated generally longer than the examples exemplified herein, which treatment has a length proportional to the length of the disease process and drug effectiveness. The doses may be single doses or multiple doses as determined by the medical practitioners and treatment courses will be repeated as necessary until diminution of the disease is achieved. Optimal dosing schedules may be calculated using measurements of drug accumulation in the body. Practitioners of ordinary skill in the art can readily determine optimum dosages, dosing methodologies, and repetition rates. Optimum dosages may vary depending on the relative potency of the antisense oligonucleotide, and can generally be determined based on values in in vitro and in vivo animal studies and clinical trials. Variations in the embodiments used may also be utilized. The amount must be effective to achieve improvement including but not limited to decreased tumor growth, or tumor size reduction or to improved survival rate or length or decreased drug resistance or other indicators as are selected as appropriate measures by those skilled in the art.

Although some antisense oligonucleotides may not completely abolish tumor cell growth in vitro, these antisense compounds may be clinically useful if they inhibit tumor growth enough to allow complementary treatments, such as chemotherapy, to be effective. The pharmaceutical compositions of the present invention therefore are administered singly or in combination with other drugs, such as cytotoxic agents, immunotoxins, alkylating agents, anti-metabolites, antitumor antibiotics and other anti-cancer drugs and treatment modalities that are known in the art. The composition is administered and dosed in accordance with good medical practice taking into account the clinical condition of the individual patient, the site and method of administration, scheduling of administration, and other factors known to medical practitioners. The “effective amount” for growth inhibition is thus determined by such considerations as are known in the art. The pharmaceutical composition may contain more than one embodiment of the present invention.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promotor to allow the sequence to be expressed in the targeted cell.

Once the oligonucleotide sequences are ready for delivery they can be introduced into cells as is known in the art. Transfection, electroporation, fusion, liposomes, colloidal polymeric particles and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

The present invention provides vectors comprising an expression control sequence operatively linked to the oligonucleotide sequences of the invention. The present invention further provides host cells, selected from suitable eucaryotic and procaryotic cells, which are transformed with these vectors as necessary. Such transformed cells allow the study of the function and the regulation of malignancy and the treatment therapy of the present invention.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either procaryotic or eucaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausub; et al, Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al., BioTechniques (1986) 4:504-512 and include, for example, stable or transient transfection, lipofection, electroporation and infection with recombinant viral vectors.

Recombinant methods known in the art can also be used to achieve the antisense inhibition of a target nucleic acid. For example, vectors containing antisense nucleic acids can be employed to express an antisense message to reduce the expression of the target nucleic acid and therefore its activity.

The present invention also provides a method of evaluating if a compound inhibits transcription or translation of an Cks1 or Skp2 gene and thereby modulates (i.e., reduces) cell proliferation comprising transfecting a cell with an expression vector comprising a nucleic acid sequence encoding Cks1 or Skp2, the necessary elements for the transcription or translation of the nucleic acid; administering a test compound; and comparing the level of expression of the Cks1 or Skp2 with the level obtained with a control in the absence of the test compound.

The present invention provides detectably labeled oligonucleotides for imaging Cks1 or Skp2 polynucleotides within a cell. Such oligonucleotides are useful for determining if gene amplification has occurred, and for assaying the expression levels in a cell or tissue using, for example, in situ hybridization as is known in the art.

While the present invention has been described with specificity in accordance with certain of its; preferred embodiments, the, following examples serve only to illustrate the invention and are not intended to limit the invention.

### EXAMPLES

### Example 1

### Antisense Inhibition of Target RNA

A. Preparation of Oligonucleotides for Transfection

A carrier molecule, comprising either a lipitoid or cholesteroid, was prepared for transfection by diluting to 0.5 mM in water, followed by sonication to produce a uniform solution, and filtration through a 0.45 μm PVDF membrane. The lipitoid or cholesteroid was then diluted into an appropriate volume of OptiMEM™ (Gibco/BRL) such that the final concentration would be approximately 1.5-2 nmol lipitoid per μg oligonucleotide.

Antisense and control oligonucleotides were prepared by first diluting to a working concentration of 100 μM in sterile Millipore water, then diluting to 2 μM (approximately 20 mg/mL) in OptiMEM™. The diluted oligonucleotides were then immediately added to the diluted lipitoid and mixed by pipetting up and down.

B. Transfection

SW620 or normal human fibroblasts cells were plated in growth media with serum at 2×10⁵ cells per well in 6-well culture dishes, and allowed to incubate overnight. The cells were then transfected by adding the oligonucleotide/lipitoid mixture, immediately after mixing, to a final concentration of 200 nM oligonucleotide. The cells were then incubated with the transfection mixture overnight at 37° C., 5% CO₂. After incubation, the transfection mixture was removed and replaced with normal growth media containing serum.

C. Total RNA Extraction and Reverse Transcription

Total RNA was extracted from the transfected cells using the RNeasy™ kit (Qiagen Corporation, Chatsworth, Calif.), following protocols provided by the manufacturer. Following extraction, the RNA was reverse-transcribed for use as a PCR template. Generally 0.2-1 μg of total extracted RNA was placed into a sterile microfuge tube, and water was added to bring the total volume to 12.5 μL. 7.5 μL of a buffer/enzyme mixture was added to each tube. The buffer/enzyme mixture was prepared by mixing, in the order listed, 2.5 μL H₂O, 2.0 μL 10× reaction buffer, 10 μL (20 pmol) oligo dT, 1.0 μL dNTP mix (10 mM each), 0.5 μL (20 u) RNAsin® (Ambion, Inc., Hialeah, Fla.) and 0.5 μL (50 u) MMLV reverse transcriptase (Ambion, Inc.). The contents of the microfuge tube were mixed by pipetting up and down, and the reaction was incubated for 1 hour at 42° C.

D. PCR Amplification and Quantification of Target Sequences

Following reverse transcription, target genes were amplified using the Roche Light Cycler™ real-time PCR machine. 20 μL aliquots of PCR amplification mixture were prepared by mixing the following components in the order listed: 2 μL 10×PCR buffer II (containing 10 mM Tris pH 8.3 and 50 mM KCl, Perkin-Elmer, Norwalk, Conn.) 3 MM MgCl₂, 140 μM each dNTP, 0.175 pmol of each Cks1 oligo, 1:50,000 dilution of SYBR® Green, 0.25 mg/mL BSA, 1 unit Taq polymerase, and H₂O to 20 μL. SYBR® Green (Molecular Probes, Eugene, Oreg.) is a dye that fluoresces when bound to double-stranded DNA, allowing the amount of PCR product produced in each reaction to be measured directly. 2 μL of completed reverse transcription reaction was added to each 20 μL aliquot of PCR amplification mixture, and amplification was carried out according to standard protocols.

Amounts of amplified target sequences obtained from each PCR reaction were normalized through comparison with an internal control (e.g., beta-actin). Table 1 below indicates that Cks1 mRNA levels were reduced in SW620 cells following transfection with Cks1 antisense oligonucleotides (SEQ ID NO:26-30), relative to control mRNAs.

TABLE 1
Antisense Cks1 Oligonucleotides Knockout
Cks1 mRNA in SW620 Cells

| Cks1 oligonucleotide | Percent mRNA |
|---|---|
| transfected | reduction |
| C39-1 | 79.0 |
| SEQ ID NO: 5 | |
| C39-2 | 85.0 |
| SEQ ID NO: 6 | |
| C39-3 | 70.0 |
| SEQ ID NO: 7 | |
| C39-4 | 49.0 |
| SEQ ID NO: 8 | |
| C39-5 | 85.0 |
| SEQ ID NO: 9 | |

### Example 2

### Soft Agar Assay

The bottom layer consisted of 2 ml of 0.6% agar in media plated fresh within a few hours of layering on the cells. For the cell layer, cells transfected as described in Example 1 were removed from the plate in 0.05% trypsin and washed twice in media. Cells were counted in coulter counter, and resuspended to 106 per ml in media. 10 ml aliquots were placed with media in 96-well plates (to check counting with WST1), or diluted further for soft agar assay. 2000 cells were plated in 800 ml 0.4% agar in duplicate wells above 0.6% agar bottom layer.

Media layer: After cell layer agar solidifies, 2 ml of media was bled on top and antisense or reverse control oligo was added without delivery vehicles. Fresh media and oligos are added every 3-4 days.

Colonies were formed in 10 days to 3 weeks. Fields of colonies were counted by eye. WST-1 metabolism values were used to compensate for small differences in starting cell number. Larger fields can be scanned for visual record of differences. SW620 cells transfected with antisense gave rise to fewer colonies compared to cells transfected with the control oligonucleotide.

Cell colonies were counted in 6 randomly-selected grids across each soft-agar well. The number of colonies was normalized by comparison with a starting WST1 value. (WST-1 Cell Proliferation Assay available from, for example, Panvera.)

Table 2 below indicate that treating SW620 cells with Cks1 antisense oligonucleotides reduced anchorage-dependent growth, relative to control cells transfected with the respective reverse complement oligonucleotides.

TABLE 2
Cks1 Antisense Oligonucleotides Inhibit
Anchorage-Dependent Growth

| | Colony number |
|---|---|
| Cks1 oligonucleotide | normalized to number |
| transfected | of starting cells |
| 39-2AS | 175 |
| 39-2RC | 340 |
| 39-5AS | 110 |
| 39-5RC | 540 |
| KRAS-AS | 165 |
| KRAS-RC | 440 |

A similar experiment was performed using Skp2 antisense-treated SW620 cells, and the results are shown in FIG. 12.

A modified protocol was used to generate the data shown in FIG. 12. In this protocol, the number of colonies was determined by Alamar blue reading and the assays were performed in 96-well format. This protocol was used to generate the date shown in FIGS. 12 and 13. The protocol is as follows:

96-well soft agar assay in poly(HEMA) coated plates. Soft agar assay monitors the ability of cells to grow in an anchorage-independent manner, a hallmark for transformed phenotype. The general design of the assay is to force cells in a 3-dimensional gel structure in a plate without the possibility of adhering to plastic, and to monitor colony formation as a read-out for growth under those conditions. A bottom layer of poly(HEMA) prevents cells from attaching to the plastic and a defined number of cells will be embedded in a top thin layer of low percentage agar. This assay is simple in set up and takes 6-7 days to completion. It is preferred to include with each set of assays a positive control known to affect growth in soft agar. It is also preferred to include the wild type untransfected cell line into the assay to discriminate between negative transfection impact versus problematic soft agar set up.

Agar Preparation

Noble agar is made to 4% in 1×PBS-CMF (about 100 ml/bottle) and autoclaved (liquid cycle, 121° C., 15 minutes). It is kept at room temperature afterwards and will become solid.

Poly(HEMA) Plates Preparation

Non-tissue culture treated 96-well plates (Costar ref # 3370) are coated with poly(2-hydroxyethyl methacrylate or poly(HEMA). Sigma ref# P3932). Poly(HEMA) is a known anti-adhesive that will prevent cells from attaching to the bottom of the plate.

Master solution of Poly(HEMA) is made as follows: 3 g powder is dissolved in 25 ml final 95% Ethanol (preferably 100% ethanol diluted, not the technical ethanol ready at 95%) in a 50 ml Falcon tube. The tube is covered with parafilm to avoid evaporation. The tube is shaken vigorously for 1-2 days at room temperature until it is fully dissolved.

A 1/10 dilution of the poly(HEMA) master solution is prepared in 95% ethanol (using 100% Ethanol). Each well of a 96-well plate is coated with 50 ul of the diluted poly(HEMA). Plates are placed on a Labsystem Wellmix shaker set at variable speed, with speed set between 5 and 6, until the plates are dry (approx. 24 h). Preferably, the coating is totally translucent. If small holes are still present in a few wells, these can be recoated with 20 ul of diluted poly(HEMA) for another 24 h.

Plates are then put in a tissue culture hood overnight under UV to sterilize. The final step is to gently wash each well twice with 200 ul of PBS 1×, using a multichannel pipette. The PBS is removed, then the plates are wrapped in Saran plastic and kept at room temperature until use.

In this assay, all steps are performed the same day. Cells on which the assay is to be run are trypsinized and counted using a hemacytometer. Dilutions are calculated so that (depending on the cell type) 350 to 700 cells/per well are seeded in 50 ul media.

In order to control for even plating, 100 ul/well of cells are seeded in a regular flat bottom plate (not poly(HEMA) treated) with the same layout as in the poly(HEMA) plate. 50 ul is then removed from each well using a multichannel pipette and moved to a poly(HEMA) plate; that plate will be transfected in about 2 hours. It is returned to the incubator for cells to recover.

For the efficiency plate (non poly(HEMA) treated) 50 ul of Alamar Blue (Trek Diagnostic Systems, ref # 00100-100) diluted 1/10 into media is added to each well. The plate is incubated at 37° C. for 24 to 36 h. Fluorescence is monitored with Excitation 530 nm and Emission at 590 nm. If seeding is even, the fluorescence values should be even. If the seeding seems uneven, normalization of the final data to these readings will have to take place.

Cell transfection. The assay set up is similar to a proliferation assay except that there is only 1 plate instead of 4. The outer edges of the plate are not used but contain media to avoid edge effects. The oligos are tested in triplicate with AS and RC for one gene set in 1 column from row B to row G The blank uses column 1 B-G It is treated identically to wild type cells except that no cells are in these wells. 11 columns are used, as in proliferation assays. Wild type (untransfected cells) are used, as well as a positive control and a negative control. 7 genes/plate remain to be tested in triplicates with AS and RC.

The type of lipid used depends on the cell type. For SW620 cells, 400 cells/well are transfected with 300 nM oligos using lipitoid 2 (L2) at 1:2.5 ratio. For each oligo only 3 wells are transfected. The final volume in each well after transfection and agar addition is 150 ul (50 ul of cells +50 ul transfection mix +50 ul agar). The transfection mix is performed in 50 ul calculated 3× so that the final concentration of oligo will be 300 nM. Once the agar gels, 100 ul of media is added on top.

For each triplicate, the following solutions are prepared in 2 Eppendorf tubes: (a) 150 ul OptiMEM +3 ul oligo; and (b) 150 ul OptiMEM +7.5 ul L2; immediately mix A into B, and pipette up and down 4-5 times, then add 50 ul of mix to the 3 appropriate wells. This operation is repeated for the next oligo. Once a column is filled with a multichannel set at 30 ul, the 6 wells are gently mixed 8-10 times in the column to make sure all cells are in a homogeneous transfection solution.

For the wild type (untransfected cells) and the blank, the following solutions are prepared in 2 Eppendorf tubes: (a) 150 ul OptiMEM; and (b) 150 ul OptiMEM. Immediately mix A into B; and pipette up and down 4-5 times, then distribute 50 ul to the appropriate wells.

Agar should be added within 2 hours. Noble agar (DIFCO) 4% is melted in the microwave oven, and when fully melted, the agar is placed at 56° C. in a waterbath for at least 10 minutes but not longer than 30 minutes. In the 96-well set up, a final concentration of agar at 0.35% is used. 12×5 ml polystyrene round bottom Falcon tubes are prepared with 840 ul media and kept in a heat block at 37° C. (37° to 40° C. is acceptable). 2×15 ml polypropylene conical Falcon tubes with 34 ml of melted 4% agar are prepared, then placed in the heat block at 60° C. (60° to 63° C. is acceptable). These tubes act as stock of concentrated agar for later dilution; the larger the volume the more inertia for keeping at melting temperature.

8 to 10 times the transfected cells of the column are gently mixed to create a homogeneous cell suspension. 300 ul of 4% agar is added into the 840 ul warm media (this makes a 1.05% agar solution) and mixed so it is homogeneous. Using a 250 ul repetitor set to distribute 50 ul, 4 times, 50 ul of identical AS is added to the 3 wells, then the leftover is discarded; the procedure is repeated for the same operation for the RC. The plate is set at room temperature for 10-15 minutes to gel firmly, then 100 ul media is added gently on all wells using a multichannel pipette. The plate is set at 37° C. for 6-7 days. After 4-5 days, colonies should be visible.

Alamar Blue staining. After 6-7 days, depending on how quickly the colonies are appearing, 20 ul undiluted Alamar blue dye is added to each well including blank. The plate is then gently shaken for 10-15 minutes to insure even penetration in the agar mesh. Then plate is returned to the incubator and fluorescence (Excitation 530 nm, Emission 590 nm) is monitored after several hours (generally, multiple readings are preferred, at about 3, 5 and 24 h). Depending on the number of cells and the transfection effect, statistical significance is reached after approximately 5 h and improves with longer incubation times.

Alamar Blue is a fluorescent dye that can be used as a growth indicator based on detection of metabolic activity. Fluorescence appears when Alamar Blue is chemically reduced in response to cell growth. The higher the number of cells, the more reduced dye is produced and detected with 530 nm Excitation, 590 nm Emission. The oxidized form of the dye does not fluoresce significantly in that range. When most of the dye is reduced, the Alamar Blue turns from blue to pink. However without any visible color changes, reduced dye may be formed.

In another experiment, SW620 cells were transfected with Skp2 antisense oligonucleotide (SEQ ID NO:15) or reverse control oligonucleotide (SEQ ID NO:16) as described above. As shown in FIG. 10A, the antisense treatment, but not the reverse control treatment, caused a reduction in Skp2 mRNA levels at both 24 and 48 hours. FIG. 10B indicates that the levels of p27Kip1 were higher in the antisense-treated cells than in the reverse control-treated cells. The Cyclin E levels remained the same as determined by comparison to the p34cdc2 protein levels which served as a gel loading control. p34cdc2 protein levels do no change during the cell cycle.

### Example 3

### Increased Release of Lactate Dehydrogenase by Antisense-Treated Cells

SW620 cells and MRC9 cells were transformed as described in Example 1 using the antisense oligonucleotide of SEQ ID NO:30 and the reverse complement (SEQ ID NO:35). The LDH assay was performed using a Roche LDH kit. As shown in FIG. 6, SEQ ID NO:30 induced greater cytotoxicity than did the corresponding reverse control oligonucleotide. Bcl-2 and Bcl-2RC are positive control oligonucleotides to validate the experimental conditions. 40-1 (SEQ ID NO:36) and RC40-1 (SEQ ID NO:41) are antisense and reverse control oligonucleotides specific for Cks2.

### Example 4

### Cell Cycle Profile of Antisense-Treated Cells

This assay was performed using the following materials: PI/RNaseA solution: added to 1 ml PBS +1% serum-20 ul PI stock and 10 ul RNaseA stock. 50×PI stock: 0.5 mg/ml propidium iodide in 38 mM Sodium Citrate pH 7.0. Stored in the refrigerator protected from light. 100×RNaseA stock: 25 mg/ml RNaseA in Tris/HCl pH 7.5, 15 mM NaCl. If made from powder, it is boiled for 15 minutes, cooled at RT, aliquoted and stored at −20° C.

1 well of a 6-well dish of mammalian cells was used as sample. Cells were harvested routinely using PBS/EDTA instead of Trysin which results in fewer dead cells and less clumping. Cells were spun at 1000 g for 5 minutes and supernatant was discarded. The pellet was washed once with PBS supplemented with 1% serum, then the cells were resuspended in 0.5 ml PBS. 10 ml EtOH (80%) was added very slowly while vortexing at level 3-5 to prevent clumping.

Cells were left in the fixative at 4° C. for at least 15 minutes, and fixed cells can be kept this way for several weeks in the refrigerator. Cells were centrifuged at 1000 g for 5 minutes, then washed with PBS supplemented with 1% serum. Cells were then resuspended in 1 ml Propidium Iodide/RNaseA solution, freshly made each time and incubated at 37° C. for 30 minutes.

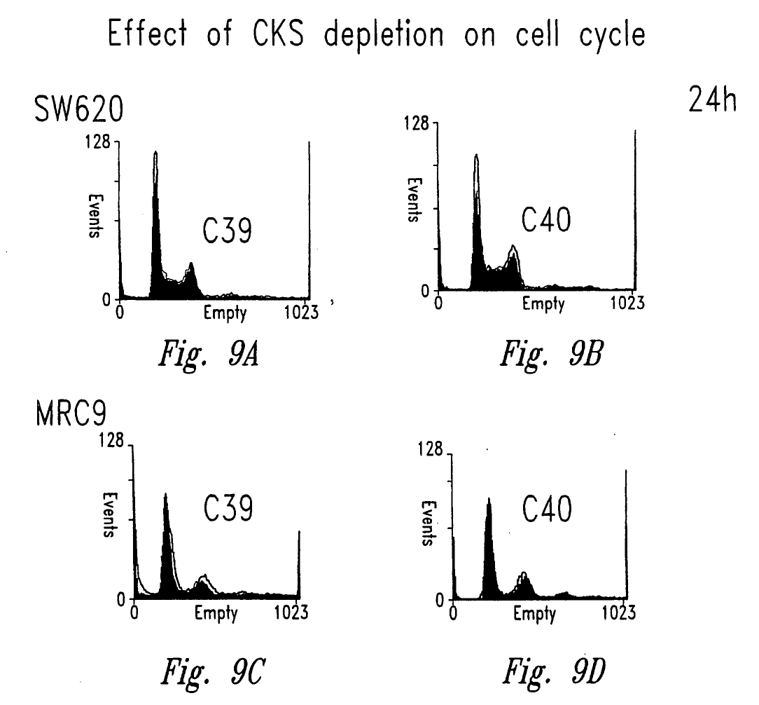

Optionally, cells are pipetted through a cell strainer into the FACS tube. The cells were analyzed within 24 hours, and cells were kept on ice and protected from light until FACS analysis. 10,000 cells were counted using FL2-A as filter. As shown in FIG. 9, Cks depletion by antisense treatment did not significantly alter the cell cycle profile of asynchronously growing SW620 and MRC9 cells.

From the foregoing it will be appreciated that, although specific embodiments of the invention have been described herein for purposes of illustration, various modifications may be made without departing from the spirit and scope of the invention.

We claim:

1. A method of treating cancer using an anti-cks-1 antisense oligonucleotide.


SEQUENCE LISTING (45 sequences)

SEQ ID NO: 1 - DNA, 717 bp, Homo sapiens
agagcgatcatgtcgcacaaacaaatttactattcggacaaatacgacgacgaggagtttgagtatcgacatgtcatgctgcccaaggacatagccaagctggtccctaaaacccatctgatgtctgaatctgaatggaggaatcttggcgttcagcagagtcagggatgggtccattatatgatccatgaaccagaacctcacatcttgctgttccggcgcccactacccaagaaaccaaagaaatgaagctggcaagctacttttcagcctcaagctttacacagctgtccttacttcctaacatctttctgataacattattatgttgccttcttgtttctcactttgatatttaaaagatgttcaatacactgtttgaatgtgctggtaactgctttgcttcttgagtagagccaccaccaccatagcccagccagatgagtgctctgtggacccacagcctaagctgagtgtgaccccagaagccacgatgtgctctgtatccagaacacacttggcagatggaggaagcatctgagtttgagaccatggctgttacagggatcatgtaaacttgctgtttttgttttttctgccgggtgttgtatgtgtggtgacttgcggatttatgtttcagtgtactggaaactttccattttattcaagaaatctgttcatgttaaaagccttgattaaagaggaagtttttataat

SEQ ID NO: 2 - protein, 79 aa, Homo sapiens [sequence omitted]

SEQ ID NO: 3 - DNA, 1462 bp, Homo sapiens
cgggctgtagagccttgcgcgcgcagtggggatggaacgttgctaggcttagcgggtctggctgctgggggcccgagcagcacgctcggagccgccgcgcgccaaagcgggaatctgggaggcgagcagctctgcagttaatgcacgtattttaaactcccgggcctgcggacgctatgcacaggaagcacctccaggagattccagacctgagtagcaacgttgccaccagcttcacgtggggatgggattccagcaagacttctgaactgctgtcaggcatgggggtctccgccctggagaaagaggagcccgacagtgagaacatcccccaggaactgctctcaaacctgggccacccggagagccccccacggaaacggctgaagagcaaagggagtgacaaagactttgtgattgtccgcaggcctaagctaaatcgagagaactttccaggtgtttcatgggactcccttccggatgagctgctcttgggaatcttttcctgtctgtgcctccctgagctgctaaaggtctctggtgtttgtaagaggtggtatcgcctagcgtctgatgagtctctatggcagaccttagacctcacaggtaaaaatctgcacccggatgtgactggtcggttgctgtctcaaggggtgattgccttccgctgcccacgatcatttatggaccaaccattggctgaacatttcagcccttttcgtgtacagcacatggacctatcgaactcagttatagaagtgtccaccctccacggcatactgtctcagtgttccaagttgcagaatctaagcctggaaggcctgcggctttcggatcccattgtcaatactctcgcaaaaaactcaaatttagtgcgacttaacctttctgggtgttctggattctctgaatttgccctgcagactttgctaagcagctgttccagactggatgagctgaacctctcctggtgttttgatttcactgaaaagcatgtacaggtggctgttgcgcatgtgtcagagaccatcacccagctgaatcttagcggctacagaaagaatctccagaaatcagatctctctactttagttagaagatgccccaatcttgtccatctagacttaagtgatagtgtcatgctaaagaatgactgctttcaggaatttttccagctcaactacctccaacacctatcactcagtcggtgctatgatataatacctgaaactttactattagtgacaagagctggggttaggatccggttggactctgacatcggatgccctcaaacatacagaacttccaaactcaagtccagccataagctattttgccaacatgtcagagtaatctgtatttttgtatgtgatttctacttttatagacttgttttaaaacaataaaacacatttttataaaaatgagtgcttaaactaaaaaaaaaaaaaaaaaaaaa

SEQ ID NO: 4 - protein, 410 aa, Homo sapiens
MHRKHLQEIPDLSSNVATSFTWGWDSSKTSELLSGMGVSALEKEEPDSENIPQELLSNLGHPESPPRKRLKSKGSDKDFVIVRRPKLNRENFPGVSWDSLPDELLLGIFSCLCLPELLKVSGVCKRWYRLASDESLWQTLDLTGKNLHPDVTGRLLSQGVIAFRCPRSFMDQPLAEHFSPFRVQHMDLSNSVIEVSTLHGILSQCSKLQNLSLEGLRLSDPIVNTLAKNSNLVRLNLSGCSGFSEFALQTLLSSCSRLDELNLSWCFDFTEKHVQVAVAHVSETITQLNLSGYRKNLQKSDLSTLVRRCPNLVHLDLSDSVMLKNDCFQEFFQLNYLQHLSLSRCYDIIPETLLLVTRAGVRIRLDSDIGCPQTYRTSKLKSSHKLFCQHVRVICIFVCDFYFYRLVLKQ

SEQ ID NO: 5 - DNA, 25 bp, Artificial Sequence
  Antisense oligonucleotide
agacagtatgccgtggagggtggac

SEQ ID NO: 6 - DNA, 25 bp, Artificial Sequence
  Antisense oligonucleotide
caggtgggaggtgccgtatgacaga

SEQ ID NO: 7 - DNA, 25 bp, Artificial Sequence
  Antisense oligonucleotide
cttcgccttccagattcccgctttg

SEQ ID NO: 8 - DNA, 25 bp, Artificial Sequence
  Antisense oligonucleotide
gtttcgcccttagaccttccgcttc

SEQ ID NO: 9 - DNA, 25 bp, Artificial Sequence
  Antisense oligonucleotide
ccccttgagacagcaaccgaccagt

SEQ ID NO: 10 - DNA, 25 bp, Artificial Sequence
  Antisense oligonucleotide
tgaccagccaacgacagagttcccc

SEQ ID NO: 11 - DNA, 25 bp, Artificial Sequence
  Antisense oligonucleotide
aggacacccaggaaggttaagtcgc

SEQ ID NO: 12 - DNA, 25 bp, Artificial Sequence
  Antisense oligonucleotide
cgctgaattggaaggacccacagga

SEQ ID NO: 13 - DNA, 25 bp, Artificial Sequence
  Antisense oligonucleotide
ggcaatggtggtgaaatgggagcaa

SEQ ID NO: 14 - DNA, 25 bp, Artificial Sequence
  Antisense oligonucleotide
aacgagggtaaagtggtggtaacgg

SEQ ID NO: 15 - DNA, 25 bp, Artificial Sequence
  Antisense oligonucleotide
gttctcactgtcgggctcctctttc

SEQ ID NO: 16 - DNA, 25 bp, Artificial Sequence
  Antisense oligonucleotide
ctttctcctcgggctgtcactcttg

SEQ ID NO: 17 - DNA, 25 bp, Artificial Sequence
  Antisense oligonucleotide
ggcaatcaccccttgagacagcaac

SEQ ID NO: 18 - DNA, 25 bp, Artificial Sequence
  Antisense oligonucleotide
caacgacagagttccccactaacgg

SEQ ID NO: 19 - DNA, 25 bp, Artificial Sequence
  Antisense oligonucleotide
tgagacagtatgccgtggagggtgg

SEQ ID NO: 20 - DNA, 25 bp, Artificial Sequence
  Antisense oligonucleotide
ggtgggaggtgccgtatgacagagt

SEQ ID NO: 21 - DNA, 24 bp, Artificial Sequence
  Antisense oligonucleotide
tgggcttttgcagtgtcagtcggc

SEQ ID NO: 22 - DNA, 24 bp, Artificial Sequence
  Antisense oligonucleotide
cggctgactgtgacgttttcgggt

SEQ ID NO: 23 - DNA, 25 bp, Artificial Sequence
  Antisense oligonucleotide
ccccatgcctgacagcagttcagaa

SEQ ID NO: 24 - DNA, 25 bp, Artificial Sequence
  Antisense oligonucleotide
aagacttgacgacagtccgtacccc

SEQ ID NO: 25 - DNA, 1600 bp, Homo sapiens
gaattccgggctgtagagccttgcgcgcgcagtggggatggaacgttgctaggcttagcgggtctggctgctgggggcccgagcagcacgctcggagccgccgcgcgccaaagcgggaatctgggaggcgagcagctctgcagttaatgcacgtattttaaactcccgggcctgcggacgctatgcacaggaagcacctccaggagattccagacctgagtagcaacgttgccaccagcttcacgtggggatgggattccagcaagacttctgaactgctgtcaggcatgggggtctccgccctggagaaagaggagcccgacagtgagaacatcccccaggaactgctctcaaacctgggccacccggagagccccccacggaaacggctgaagagcaaagggagtgacaaagactttgtgattgtccgcaggcctaagctaaatcgagagaactttccaggtgtttcatgggactcccttccggatgagctgctcttgggaatcttttcctgtctgtgcctccctgagctgctaaaggtctctggtgtttgtaagaggtggtatcgcctagcgtctgatgagtctctatggcagaccttagacctcacaggtaaaaatctgcacccggatgtgactggtcggttgctgtctcaaggggtgattgccttccgctgcccacgatcatttatggaccaaccattggctgaacatttcagcccttttcgtgtacagcacatggacctatcgaactcagttatagaagtgtccaccctccacggcatactgtctcagtgttccaagttgcagaatctaagcctggaaggcctgcggctttcggatcccattgtcaatactctcgcaaaaaactcaaatttagtgcgacttaacctttctgggtgttctggattctctgaatttgccctgcagactttgctaagcagctgttccagactggatgagctgaacctctcctggtgttttgatttcactgaaaagcatgtacaggtggctgttgcgcatgtgtcagagaccatcacccagctgaatcttagcggctacagaaagaatctccagaaatcagatctctctactttagttagaagatgccccaatcttgtccatctagacttaagtgatagtgtcatgctaaagaatgactgctttcaggaatttttccagctcaactacctccaacacctatcactcagtcggtgctatgatataatacctgaaactttacttgaacttggagaaattcccacactaaaaacactacaagtttttggaatcgtgccagatggtacccttcaactgttaaaggaagcccttcctcatctacagattaattgctcccatttcaccaccattgccaggccaactattggcaacaaaaagaaccaggagatatggggcatcaaatgccgactgacactgcaaaagcccagttgtctatgaagtatttattgcaggatggtgtctcttctttagaacagggaaaataggcaggaagcccaattgctggagtacttagctagttttattcttggttttccctttgccttcattctgcaagtatactagggagccatttgagaggg

SEQ ID NO: 26 - DNA, 25 bp, Artificial Sequence
  Antisense oligonucleotide
ctgctgaacgccaagattcctccat

SEQ ID NO: 27 - DNA, 24 bp, Artificial Sequence
  Antisense oligonucleotide
accagcttggctatgtccttgggc

SEQ ID NO: 28 - DNA, 24 bp, Artificial Sequence
  Antisense oligonucleotide
cccatccctgactctgctgaacgc

SEQ ID NO: 29 - DNA, 24 bp, Artificial Sequence
  Antisense oligonucleotide
tttggtttcttgggtagtgggcgc

SEQ ID NO: 30 - DNA, 25 bp, Artificial Sequence
  Antisense oligonucleotide
actcatctggctgggctatggtggt

SEQ ID NO: 31 - DNA, 25 bp, Artificial Sequence
  Control oligonucleotide
tacctccttagaaccgcaagtcgtc

SEQ ID NO: 32 - DNA, 24 bp, Artificial Sequence
  Control oligonucleotide
cgggttcctgtatcggttcgacca

SEQ ID NO: 33 - DNA, 24 bp, Artificial Sequence
  Control oligonucleotide
cgcaagtcgtctcagtccctaccc

SEQ ID NO: 34 - DNA, 24 bp, Artificial Sequence
  Control oligonucleotide
cgcgggtgatgggttctttggttt

SEQ ID NO: 35 - DNA, 25 bp, Artificial Sequence
  Control oligonucleotide
tggtggtatcgggtcggtctactca

SEQ ID NO: 36 - DNA, 25 bp, Artificial Sequence
  Antisense oligonucleotide
gaacccagcctagactctgttggac

SEQ ID NO: 37 - DNA, 23 bp, Artificial Sequence
  Antisense oligonucleotide
cccaggcaacaactcgccggaga

SEQ ID NO: 38 - DNA, 24 bp, Artificial Sequence
  Antisense oligonucleotide
gccggtactcgtagtgttcgtcga

SEQ ID NO: 39 - DNA, 24 bp, Artificial Sequence
  Antisense oligonucleotide
tctgcttgtgggccatcctcgtgg

SEQ ID NO: 40 - DNA, 23 bp, Artificial Sequence
  Antisense oligonucleotide
gcgcagcagacaaaaccacgtcc

SEQ ID NO: 41 - DNA, 25 bp, Artificial Sequence
  Antisense oligonucleotide
cacaggttgtctcagatccgaccca

SEQ ID NO: 42 - DNA, 23 bp, Artificial Sequence
  Antisense oligonucleotide
agaggccgctcaacaacggaccc

SEQ ID NO: 43 - DNA, 24 bp, Artificial Sequence
  Antisense oligonucleotide
agctgcttgtgatgctcatggccg

SEQ ID NO: 44 - DNA, 24 bp, Artificial Sequence
  Antisense oligonucleotide
ggtgctcctaccgggtgttcgtct

SEQ ID NO: 45 - DNA, 23 bp, Artificial Sequence
  Antisense oligonucleotide
cctgcaccaaaacagacgacgcg